(19) United States
(12) Patent Application Publication
(10) Pub. No.: US 2025/0061227 A1
(43) Pub. Date: Feb. 20, 2025

(54) DISTRIBUTED STORAGE OF GENOMIC DATA

(71) Applicant: IGENTIFY LTD., Caesarea (IL)

(72) Inventors: Ellie PORTUGALI, Zichron Yaakov (IL); Zohar SHERMAN, Kerem Maharal (IL)

(21) Appl. No.: 18/723,430

(22) PCT Filed: Dec. 15, 2022
(86) PCT No.: PCT/IL2022/051329

§ 371 Date: Jun. 23, 2024

Related U.S. Application Data
(60) Provisional application No. 63/292,514, filed on Dec. 22, 2021.

(51) Int. Cl. G06F 21/62; G06F 16/25; G16H 10/60

(52) U.S. Cl. CPC G06F 21/6245; G06F 16/258; G16H 10/60

(57) ABSTRACT

A computerized data-storage system performs the following method: (a) receive a set of genomic data, comprising first items of loci-specific information. Each first item comprises encoded sequence(s) and/or item(s) of sequence metadata, and is associated with item(s) of identification information. (b) store, in first storage location(s), the set. (c) store, in a mapping storage, a mapping between the item(s) of identification information and the corresponding first item. (d) store, in second storage location(s), the item(s) of identification information, associated with individual instance(s) of an organism. The first and second storage locations are not identical. This facilitates a reconstruction, by a data-retrieval system, of at least a portion of an individual-specific instance of the set. The instance comprises a sub-set of the plurality of first items. This facilitates an enhanced level of security of the individual-specific instance of the set of genomic data.

### TECHNICAL FIELD

The presently disclosed subject matter relates to data storage, in particular storage of genomic data.

### BACKGROUND

Completed in 2003, the Human Genome Project was the first systematic attempt at decoding the whole human genome. The Project cost roughly $2.7 billion and took over a decade. Fewer than two decades later, the cost to sequence a human genome has dropped to less than $600 and, according to industry experts, should drop to less than $100 in the next five years. These lower price points have acted as a catalyst in driving the adoption of genomic medicine and has led to the genomic revolution.

Forecasts by industry experts mention clinical adoption of next generation DNA sequencing (NGS) will drive volumes from ˜5 to 7 million in 2021 to more than 100 million by 2024. If the volumes of genetic testing utilizing the micro-array technology are included, the total number of people who would benefit from this genomic and precision medicine revolution would at least be a few hundreds of millions in the next few years.

The above information is from Brett Winton, Genomics Innovation: A Catalyst For Growth-Health Care in the Genomic Age, ARK Invest, (9 Jul. 2020).

This future promise is threatened by issues of genomic data privacy, data ownership, and data security. Today, the genomic data acquired from genetic testing, is typically centralized, and stored at genetic institutes, laboratories, healthcare systems, hospitals, or other healthcare institutions, making them in control of patients' genomic data. In some examples, reports are generated based on these tests, providing the requestor with information concerning specific clinical indications, e.g. specific diseases or predisposition to diseases, drug response etc.

These institutions can leverage this data to make financial gains by either selling or licensing it. Moreover, this centralized genomic data storage approach makes the data vulnerable to data-breaches and cyber-attacks. Another problem with centralized genomic data storage at healthcare institutions is that, once a patient moves from one healthcare institution to another, there are no means that a patient can carry their genome data with them.

Various solutions are being developed, utilizing disparate encryption and masking algorithms, to ensure data privacy and security. A few exemplary publications U.S. Pat. Nos. 9,524,392, 10,013,575 mention such approaches.

Some solutions discuss solving the above-mentioned problems by distributed genomic data storage. US20210271982 discloses a method of storing, in a distributed manner, genomic information in a plurality of nodes, each containing a block chain composed of blocks connected to each other.

### GENERAL DESCRIPTION

According to a first aspect of the presently disclosed subject matter there is presented a computerized method, capable of being performed by a computerized data-storage system comprising a processing circuitry, the method comprising performing the following actions:
a) receive a set of genomic data, comprising a plurality of first items of loci-specific information,
wherein each first item of loci-specific information of the plurality of items of loci-specific information comprises at least one of one or more encoded sequences and one or more items of sequence metadata,
wherein each first item of loci-specific information is associated with at least one item of identification information;
b) store, in at least one first storage location, the set of genomic data;
c) store, in a mapping storage, a mapping between the at least one item of identification information and the corresponding first item of loci-specific information; and
d) store, in at least one second storage location, the at least one item of identification information, where the at least one second storage location is associated with at least one individual instance of an organism,
wherein the at least one first storage location and the at least one second storage location are not identical;
the method thereby facilitating a reconstruction, of at least a portion of an individual-specific instance of the set of genomic data,
the reconstruction being performed by a computerized data-retrieval system,
the individual-specific instance of the set being associated with at least one individual instance of the organism, the individual-specific instance of the set comprising a sub-set of the plurality of first items of loci-specific information,
the method thereby facilitating an enhanced level of security of the individual-specific instance of the set of genomic data, as compared to a second level of security provided in a case of performing storage, in a single location, of the individual-specific instance of the set of genomic data and of the at least one item of identification information.

In addition to the above features, the method according to this aspect of the presently disclosed subject matter can include one or more of features (i) to (xxxii) listed below, in any desired combination or permutation which is technically possible:
(i) the reconstruction comprises performing the following method:
d) receive the at least one item of identification information, from the at least one second storage location;
e) receive at least a portion of the set of genomic data, from the first storage location;
f) identify each first item of loci-specific information, based on the at least one item of identification information and on the mapping; and
g) output the at least the portion of the individual-specific instance of the set of genomic data.
(ii) the one or more encoded sequences comprising encoded sequences indicative of deviations from one or more genomic references.
(iii) the set of genomic data further comprising the one or more genomic references.
(iv) the receiving of the at least a portion of the set of genomic data utilizes the received at least one item of identification information.
(v) said step (c) further comprising storing, in a second mapping storage, a second mapping between the at least one item of identification information and at least one clinical indication.
(vi) in said step (d) the receiving of the at least one item of identification information further comprises performing the following steps:
(1) receiving clinical indication information, indicative of at least one clinical indication associated with the individual instance of the organism;
(2) identifying, based on the received clinical indication information and on the second mapping, a corresponding at least one item of identification information;
(3) obtaining a relevant at least one item of identification information, from the received of the at least one item of identification information, based on the corresponding at least one item of identification information, the relevant at least one item of identification information constituting the at least one item of identification information.
(vii) the stored mapping maps the at least one item of identification information to at least one of: the corresponding first item of loci-specific information; the one or more encoded sequences; the one or more items of sequence metadata; a pointer to the corresponding first item of loci-specific information; at least one other item of identification information.
(viii) the receiving the at least a portion of the set of genomic data, from the first storage location, comprises receiving the set of genomic data.
(ix) the receiving the at least one item of identification information associated with the at least one individual instance of the organism, from the at least one second storage location, comprises receiving all items of identification information associated with the at least one individual instance of an organism.
(x) the at least one item of identification information comprises at least one identification code.
(xi) the at least one item of identification information comprises at least one of a hash and an encoded id.
(xii) the mapping storage and the first storage location are located at the same location.
(xiii) at least some encoded sequences are of lengths different from each other.
(xiv) the organism is one of a unicellular organism, a multicellular organism and a virus.
(xv) the organism is a human.
(xvi) the organism is a proband.
(xvii) the set of genomic data comprises genetic sequence data corresponding to an entire genome of the organism.
(xviii) the method further comprises performing, prior to said step (a), the following steps to generate the set of data:
h) receiving information indicative of a raw set of genomic data
i) analyzing features of the received information;
j) extracting one or more features from the received information; and
k) encoding the one or more features, thereby generating the each first item of loci-specific information and the at least one item of identification information.
(xix) the information indicative of a raw set of genomic data is a genetic testing machine output associated with the individual instance of the organism,
the method therefore facilitating re-use of the results for other clinical needs.
(xx) the genetic testing machine output is at least one of: a proprietary binary, a proprietary text, Comma delimited, tab delimited, a Variant Call Format (VCF) file, a genotype calling file, a FastQ format file, a stream of data, or other.
(xxi) in said step (i) the analyzing of the features is based on a first knowledge corpus associated with the set of genomic data.
(xxii) the features comprise at least one of: the encoding sequence; Quality Score (QC) data associated with a locus; epigenetic data; vendor specific information.
(xxiii) the method further comprising performing, prior to said step (c):
l) encapsulating a collection of personal keys, comprising the at least one item of identification information,
wherein the storing of the at least one item of identification information comprising storing the collection of personal keys.
(xxiv) the enhanced level of security comprises a lack of direct access of external systems to the individual-specific instance of the set of genomic data.
(xxv) the method thereby facilitating access to the at least the portion of the individual-specific instance of the set of genomic data, while utilizing a reduced storage amount, as compared to a second storage amount required in a case of performing storage, in a single location, of individual-specific instances of the set of genomic data for each individual organism of a plurality of organisms.
(xxvi) a particular item of loci-specific information is associated with a single item of identification information.
(xxvii) a particular item of loci-specific information is associated with a plurality of items of identification information.
(xxviii) the at least one second storage location is associated with more than one individual instance of an organism,
wherein each item of identification information is associated with a corresponding individual instance of the more than one individual instance of the organism,
wherein the each item of identification information is associated with an identification indication that is indicative of the corresponding individual instance of the organism,
thereby facilitating the reconstruction of the at least the portion of the individual-specific instance of the set of genomic data in correspondence to the corresponding individual instance.
(xxix) the identification indication is an identification number.
(xxx) the at least one second storage location comprises at least one storage device associated with the organism.
(xxxi) the at least one storage device is one of: local storage or on-line storage.
(xxxii) the at least one item of identification information is stored in one or more personal data key files at the at least one second storage location.

According to a second aspect of the presently disclosed subject matter there is presented a computerized method, capable of being performed by a computerized data-retrieval system comprising a processing circuitry, the method comprising performing the following actions:
a) provide a set of genomic data, comprising a plurality of first items of loci-specific information,
wherein each first item of loci-specific information of the plurality of items of loci-specific information comprises at least one of an encoded sequence and sequence metadata,
wherein each first item of loci-specific information is associated with at least one item of identification information;
wherein the set of genomic data was generated by performance of the following method by a computerized data-storage system:
(i) store, in at least one first storage location, the set of genomic data;
(ii) store, in a mapping storage, a mapping between the at least one item of identification information and the each first item of loci-specific information; and
(iii) store, in at least one second storage location, the at least one item of identification information, where each second storage location is associated with at least one individual instance of an organism,
wherein the at least one first storage location and the at least one second storage location are not identical;
b) reconstruct at least a portion of an individual-specific instance of the set of genomic data,
the individual-specific instance being associated with the at least one individual instance of an organism, the individual-specific instance comprising a sub-set of a plurality of first items of loci-specific information,
wherein the reconstruction comprises the following method:
(i) receive at least a portion of the set of genomic data, from the first storage location;
(ii) receive the at least one item of identification information, from the at least one second storage location;
(iii) identify the each first item of loci-specific information, based on the at least one item of identification information and on the mapping; and
(iv) output the at least the portion of the individual-specific instance of the set of genomic data,
the method thereby facilitating an enhanced level of security of the individual-specific instance of the set of genomic data, as compared to a second level of security provided in a case of a reconstruction based on performing storage, in a single location, of the individual-specific instance of the set of genomic data and of the at least one item of identification information.

The second aspect of the disclosed subject matter can optionally include one or more of features (i) to (xxxii) listed above, mutatis mutandis, in any desired combination or permutation which is technically possible.

In addition to the above features, the method according to this aspect of the presently disclosed subject matter can include one or more of features (xxxiii) to (xxxv) listed below, in any desired combination or permutation which is technically possible:
(xxxiii) the one or more encoded sequences comprising encoded sequences indicative of deviations from one or more genomic references.
(xxxiv) the identifying the at least one first item of loci-specific information,
   based on the at least one item of identification information and on the mapping, comprises performing a lookup of the at least one item of identification information.
(xxxv) the method further comprising performing the following:
(v) responsive to the outputting of the at least the portion of the individual-specific instance of the set of genomic data, delete the reconstructed individual-specific instance of the set of genomic data from the computerized data-interpretation system.

According to a third aspect of the presently disclosed subject matter there is presented a computerized method, capable of being performed by a computerized data-interpretation system comprising a processing circuitry, the method comprising performing the following actions:
(A) receive the output of the at least the portion of the individual-specific instance of the set of genomic data, generated by the computerized data-retrieval system of the second aspect; and
(B) derive at least one item of interpretive information associated with the individual instance of the organism, based at least on the reconstructed at least a portion of the individual-specific instance of the set of genomic data.

The second aspect of the disclosed subject matter can optionally include one or more of features (i) to (xxxv) listed above, mutatis mutandis, in any desired combination or permutation which is technically possible.

In addition to the above features, the method according to this aspect of the presently disclosed subject matter can include one or more of features (xxxvi) to (xlv) listed below, in any desired combination or permutation which is technically possible:
(xxxvi) the at least one item of interpretive information is indicative of at least one clinical indication associated with the individual instance of the organism.
(xxxvii) the deriving of the interpretive information is based on a second knowledge corpus.
(xxxviii) the method further comprising performing the following:
(C) output the at least one item of interpretive information to at least one external system.
(xxxix) the output of the at least one item of interpretive information comprises a report.
(xl) the at least one of external system is associated with at least one of a physician, a genetic counselor, a health care system, a genetic test laboratory, an employer, and an insurer.
(xli) the method further comprising performing the following:
(D) responsive to one of the deriving of the interpretive information and the outputting of the at least one item of interpretive information, delete the reconstructed individual-specific instance of the set of genomic data from at least one of the computerized data-retrieval system and the computerized data-interpretation system.
(xlii) the outputting of the at least one item of interpretive information to the external system in said step (c) is performed in response to receipt of an authorization indication which indicates that the at least one external system is authorized to receive the at least one item of interpretive information.
(xliii) the authorization indication is associated with the at least one individual instance of the organism.
(xliv) the authorization indication is indicative of consent of the individual instance of the organism.
(xlv) the authorization indication is a configurable parameter.

According to a fourth aspect of the presently disclosed subject matter there is provided a computerized data-storage system, comprising a processing circuitry, configured to perform the method of the first aspect of the disclosed subject matter.

The fourth aspect of the disclosed subject matter can optionally include one or more of features (i) to (xxxii) listed above, mutatis mutandis, in any desired combination or permutation which is technically possible.

According to a fifth aspect of the presently disclosed subject matter there is provided a computerized data-retrieval system, comprising a processing circuitry, configured to perform the configured to perform the method of the second aspect of the disclosed subject matter.

The fifth aspect of the disclosed subject matter can optionally include one or more of features (i) to (xxxv) listed above, mutatis mutandis, in any desired combination or permutation which is technically possible.

According to a sixth aspect of the presently disclosed subject matter there is provided a computerized data-interpretation system, comprising a processing circuitry, configured to perform the method of the third aspect of the disclosed subject matter.

The sixth aspect of the disclosed subject matter can optionally include one or more of features (i) to (xlv) listed above, mutatis mutandis, in any desired combination or permutation which is technically possible.

According to a seventh aspect of the presently disclosed subject matter there is provided a non-transitory computer readable storage medium tangibly embodying a program of instructions that when executed by a computer, cause the computer to perform the method of any one of the first to third aspects of the disclosed subject matter.

The non-transitory computer readable storage media, disclosed herein according to this seventh aspect, can optionally further comprise one or more of features (i) to (xlv) listed above, mutatis mutandis, in any technically possible combination or permutation.

### BRIEF DESCRIPTION OF THE DRA WINGS

In order to understand the invention and to see how it can be carried out in practice, embodiments will be described, by way of non-limiting examples, with reference to the accompanying drawings, in which:

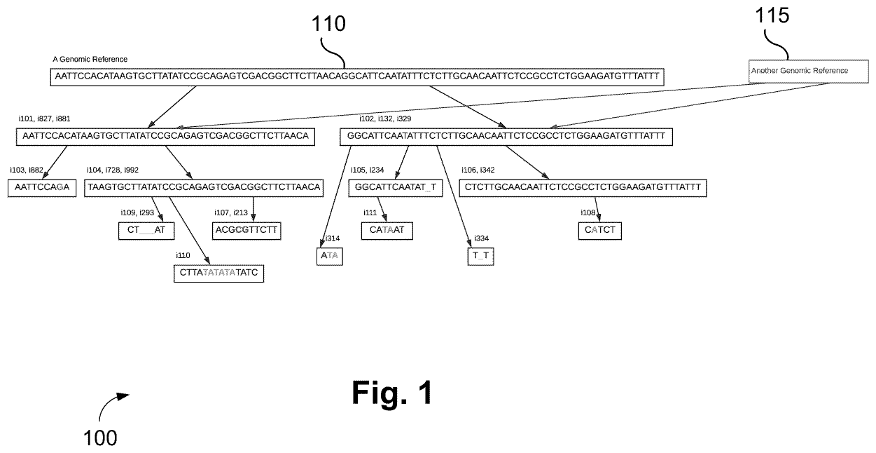

FIG. 1 illustrates schematically an example generalized view of a structure of genomic data, in accordance with some embodiments of the presently disclosed subject matter;

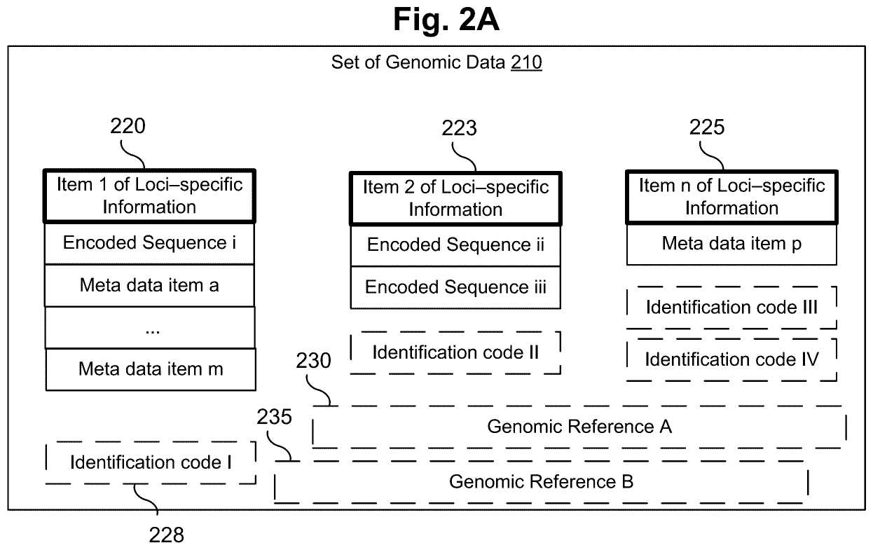

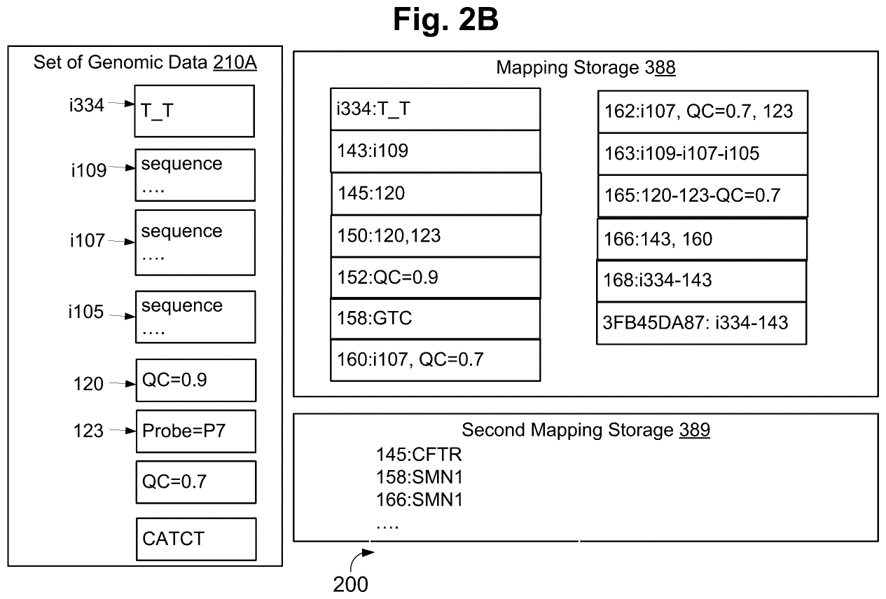

FIG. 2A illustrates schematically an example generalized view of a set of genomic data, in accordance with some embodiments of the presently disclosed subject matter;

FIG. 2B illustrates schematically an example generalized view of mapping, in accordance with some embodiments of the presently disclosed subject matter;

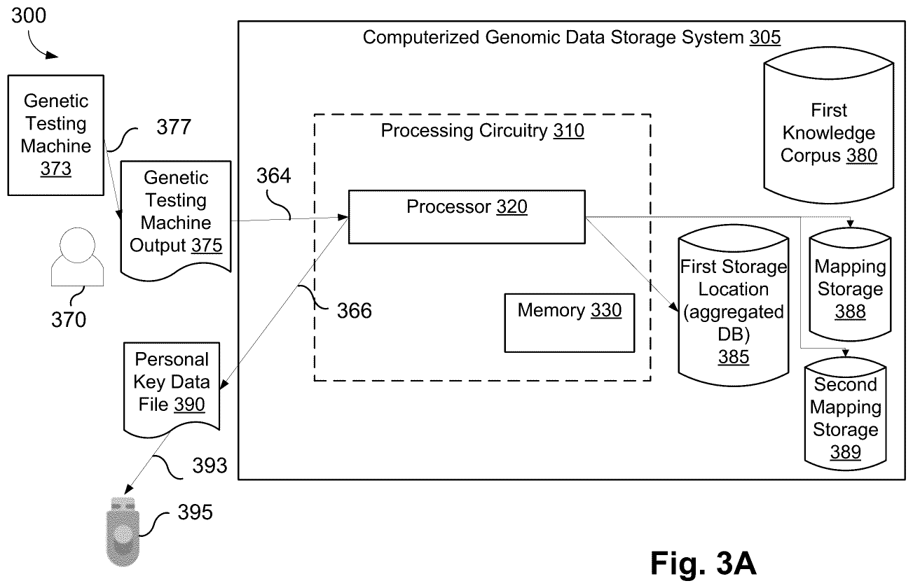

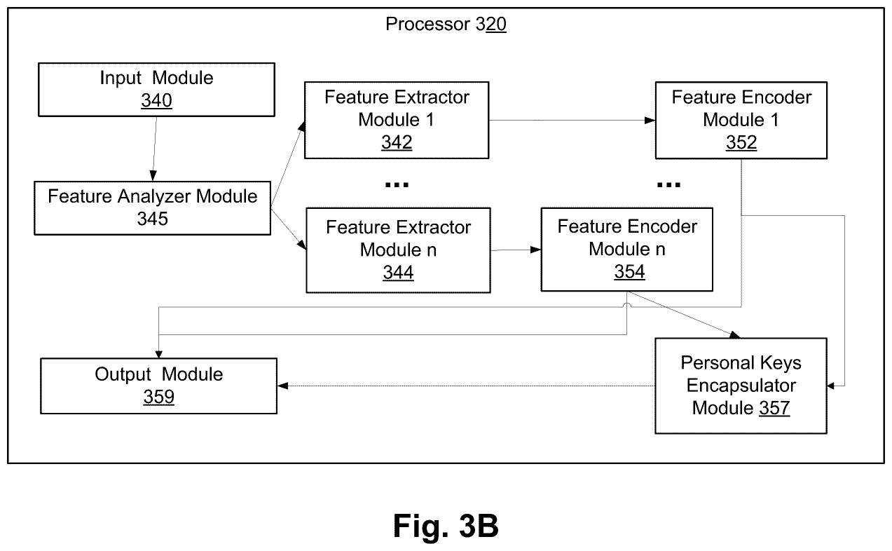

FIG. 3A illustrates schematically an example generalized schematic diagram comprising a computerized genomic data storage system, in accordance with some embodiments of the presently disclosed subject matter;

FIG. 3B illustrates schematically an example generalized schematic diagram of a processor, in accordance with some embodiments of the presently disclosed subject matter;

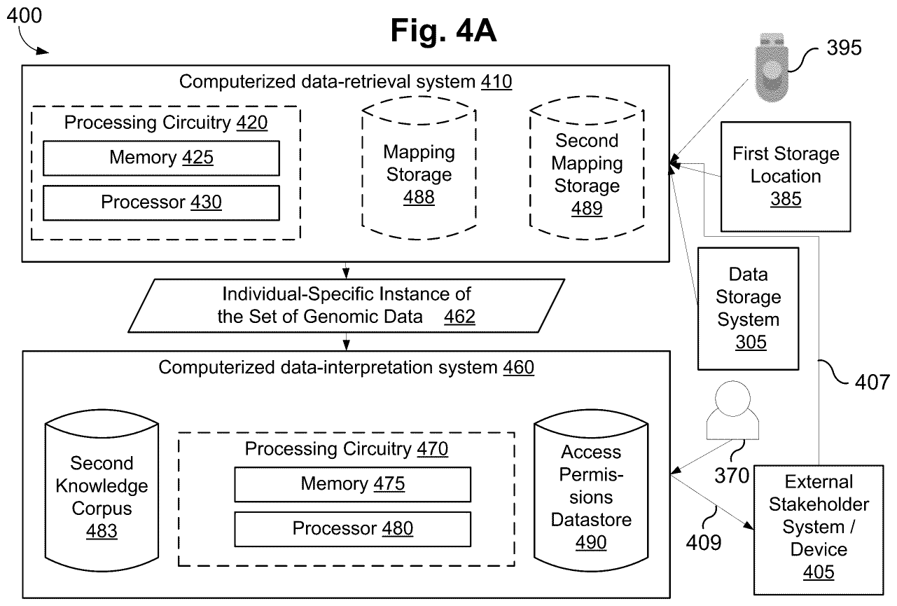

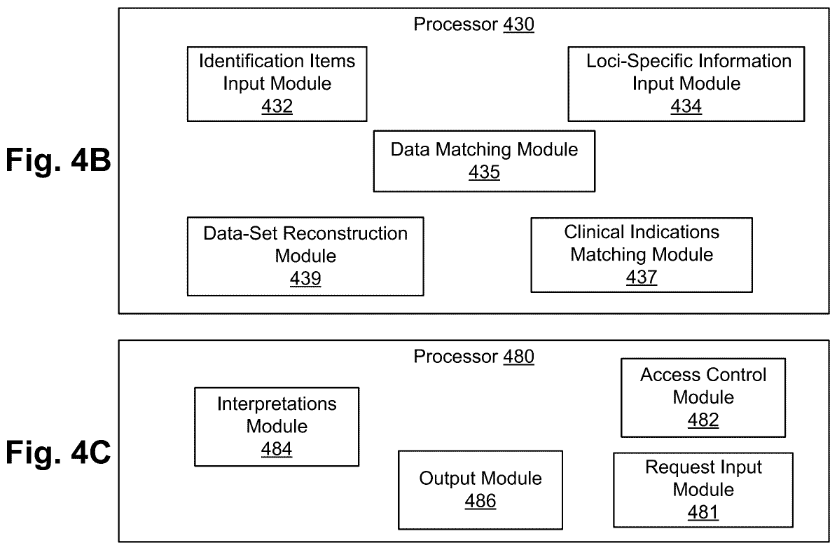

FIG. 4A schematically illustrates an example generalized schematic diagram of data retrieval and interpretation systems, in accordance with some embodiments of the presently disclosed subject matter;

FIG. 4B schematically illustrates an example generalized schematic diagram of a processor, in accordance with some embodiments of the presently disclosed subject matter;

FIG. 4C schematically illustrates an example generalized schematic diagram of a processor, in accordance with some embodiments of the presently disclosed subject matter; and

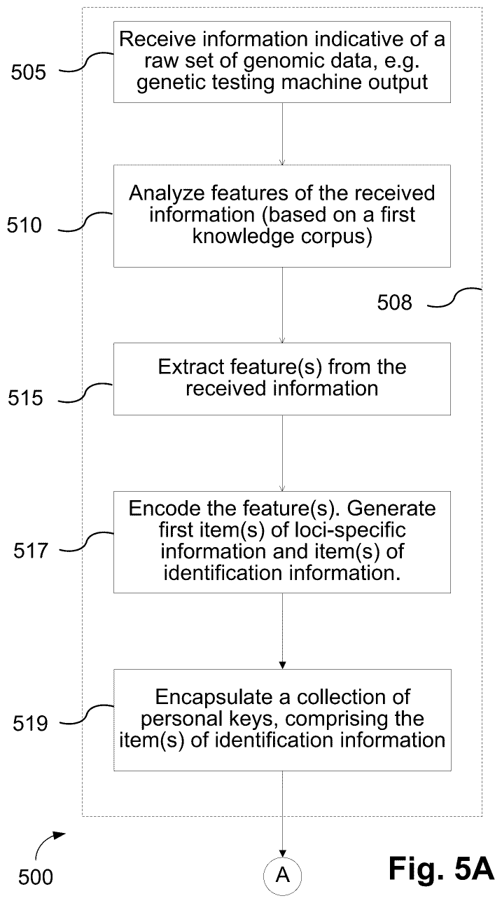

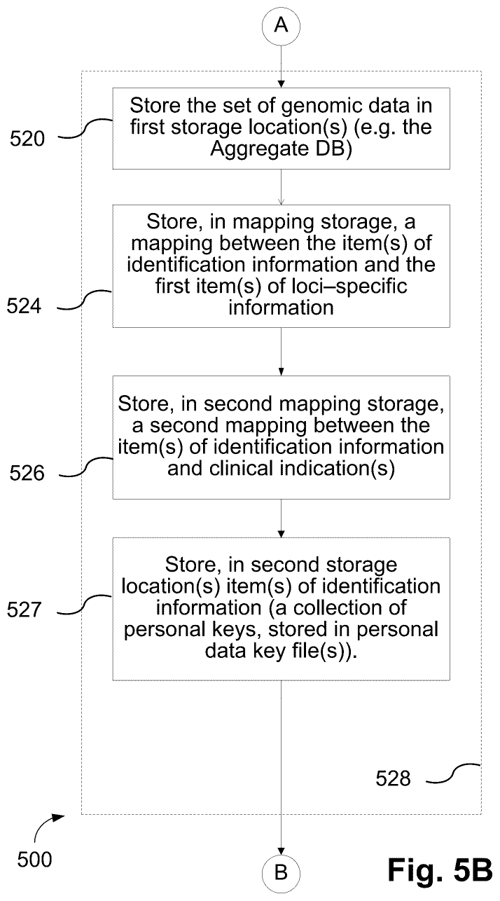

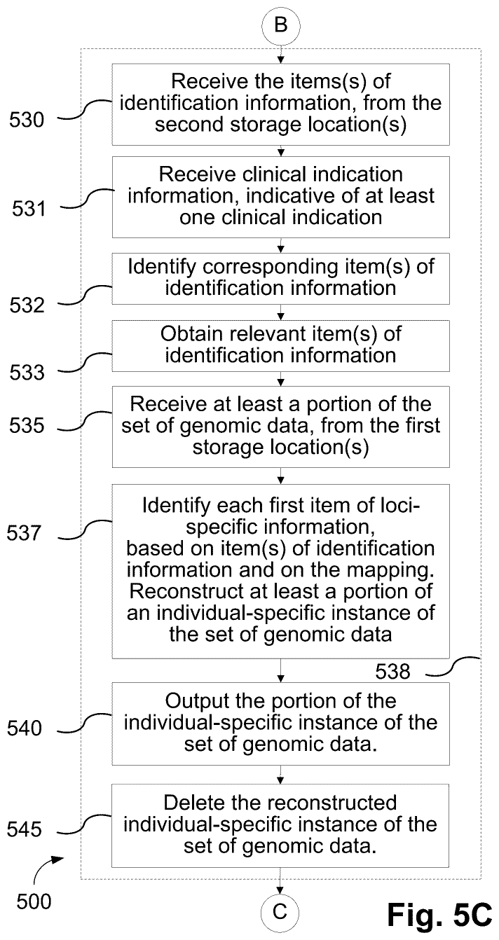

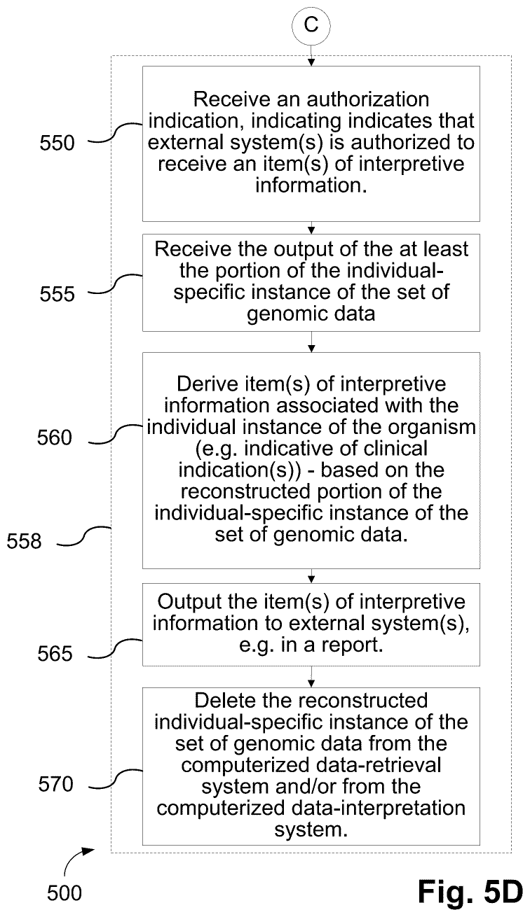

FIGS. 5A to 5D schematically illustrate one example generalized flow chart diagram, of a flow of a process or method, for retrieval and interpretation of genomic data, in accordance with some embodiments of the presently disclosed subject matter.

### DETAILED DESCRIPTION

In the drawings and descriptions set forth, identical reference numerals indicate those components that are common to different embodiments or configurations.

In the following detailed description, numerous specific details are set forth in order to provide a thorough understanding of the invention. However, it will be understood by those skilled in the art that the presently disclosed subject matter may be practiced without these specific details. In other instances, well-known methods, procedures, components and circuits have not been described in detail so as not to obscure the presently disclosed subject matter.

It is to be understood that the invention is not limited in its application to the details set forth in the description contained herein or illustrated in the drawings. The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

It will also be understood that the system according to the invention may be, at least partly, implemented on a suitably programmed computer. Likewise, the invention contemplates a computer program being readable by a computer for executing the method of the invention. The invention further contemplates a non-transitory computer-readable memory tangibly embodying a program of instructions executable by the computer for executing the method of the invention.

Those skilled in the art will readily appreciate that various modifications and changes can be applied to the embodiments of the invention as hereinbefore described without departing from its scope, defined in and by the appended claims.

Unless specifically stated otherwise, as apparent from the following discussions, it is appreciated that throughout the specification discussions utilizing terms such as “providing”, “presenting”, “receiving”, “performing”, “checking”, “recording”, “detecting”, “generating”, “setting” or the like, refer to the action(s) and/or process(es) of a computer that manipulate and/or transform data into other data, said data represented as physical, e.g. such as electronic or mechanical quantities, and/or said data representing the physical objects. The term “computer” should be expansively construed to cover any kind of hardware-based electronic device with data processing capabilities including a personal computer, a server, a computing system, a communication device, a processor or processing unit (e.g. digital signal processor (DSP), a microcontroller, a microprocessor, a field programmable gate array (FPGA), an application specific integrated circuit (ASIC), etc.), and any other electronic computing device, including, by way of non-limiting example, computerized systems 305, 410, 460 and processing circuitries 310, 420, 470 disclosed in the present application.

The operations in accordance with the teachings herein may be performed by a computer specially constructed for the desired purposes, or by a general-purpose computer specially configured for the desired purpose by a computer program stored in a non-transitory computer-readable storage medium.

Embodiments of the presently disclosed subject matter are not described with reference to any particular programming language. It will be appreciated that a variety of programming languages may be used to implement the teachings of the presently disclosed subject matter as described herein.

The terms “non-transitory memory” and “non-transitory storage medium” used herein should be expansively construed to cover any volatile or non-volatile computer memory suitable to the presently disclosed subject matter.

As used herein, the phrase “for example,” “such as”, “for instance” and variants thereof describe non-limiting embodiments of the presently disclosed subject matter. Reference in the specification to “one case”, “some cases”, “other cases”, “one example”, “some examples”, “other examples”, or variants thereof, means that a particular described method, procedure, component, structure, feature or characteristic described in connection with the embodiment(s) is included in at least one embodiment of the presently disclosed subject matter, but not necessarily in all embodiments. The appearance of the same term does not necessarily refer to the same embodiment(s) or example(s).

Usage of conditional language, such as “may”, “might”, or variants thereof, should be construed as conveying that one or more examples of the subject matter may include, while one or more other examples of the subject matter may not necessarily include, certain methods, procedures, components and features. Thus such conditional language is not generally intended to imply that a particular described method, procedure, component or circuit is necessarily included in all examples of the subject matter. Moreover, the usage of non-conditional language does not necessarily imply that a particular described method, procedure, component or circuit is necessarily included in all examples of the subject matter.

It is appreciated that certain embodiments, methods, procedures, components or features of the presently disclosed subject matter, which are, for clarity, described in the context of separate embodiments or examples, may also be provided in combination in a single embodiment or examples. Conversely, various embodiments, methods, procedures, components or features of the presently disclosed subject matter, which are, for brevity, described in the context of a single embodiment, may also be provided separately or in any suitable sub-combination.

It should also be noted that each of the figures herein, and the text discussion of each figure, describe one aspect of the presently disclosed subject matter in an informative manner only, by way of non-limiting example, for clarity of explanation only. It will be understood that the teachings of the presently disclosed subject matter are not bound by what is described with reference to any of the figures or described in other documents referenced in this application.

Bearing this in mind, attention is drawn to FIG. 1, schematically illustrating an example generalized view of a structure of genomic data, in accordance with some embodiments of the presently disclosed subject matter. Example structure 100 depicts a portion of genomic data for an individual, e.g. a person Bob, for example a portion of an encoded sequence such as a chromosome, located at particular locus/position on the chromosome. Genomic data refers here to any representation of sequences of genomic material, whether for encoding or encoding portions of the individual's genome.

Before continuing with exposition of FIG. 1, there is disclosed some example disadvantages of some existing storage methods of genomic information. In at least some examples of prior art methods, the data of a proband or other individual, e.g. the genomic data obtained from genomic or genetic testing, is stored, for example in the computers of the testing lab or of the hospital or other health institution, and the data is associated with the identification of the tested proband. This leads to at least certain disadvantages or problems.

Firstly, there are privacy and security issues. For example, the data owner (the proband, patient etc., whose genomic information is being stored) has no control over the data—it all resides at the testing or healthcare facility. He cannot “take” the data with him to show it to another institution, and he has no control over how it is used. Also, stakeholders such as doctors, testing labs, hospitals etc. have the capability to potentially abuse the user data. One example of this is trading proband data with other institutions and other parties, without the informed consent of the proband or other data owner.

Another example is that genomic data, other than that for which consent was obtained, can be viewed and processed, thus reducing the rights of the proband and violating their privacy rights. One illustrative example of this latter issue is that Bob, undergoing genetic testing related to heart disease, provided to the testing lab consent for use of his heart-disease related data, but his genomic data, stored at the lab, also includes data relevant to e.g. cancer, mental health issues, or baldness, for which he did not provide consent regarding access. This additional information is not related to the purpose for which the institution was given access to the individual's genomic material or genomic data. One non-limiting example to illustrate the problematic nature of such a situation is as follows: Bob did pre-conception screening, and, at a later date his new employer tried to access the data at the institution. The employer wants to see if he has e.g. cardiac problems, which will affect his ability to do the job, or which will increase the likelihood that he will claim disability in the future.

A further example of security issues is that if a hacker breaks into a lab's or hospital's computer, he would have access to Bob's individual data, and he would also know that the data is that of Bob specifically.

Secondly, in some cases there are capacity issues. In some examples, a full genome of a human requires more than 120 gigabytes (GB) of storage. If data of thousands (or more) clients are stored in a computer, this may require a huge amount of storage capacity. This is despite the fact that a considerable portion of genomic data is of identical value across among many individuals. Also, if an individual such as Bob wishes to have a copy of his genomic data, for storage at home, and to perhaps carry to another institution, this individual would require data storage of a size such as at least 120 gigabytes (GB). Thus in many cases it is not feasible for the individual to keep, in their possession and control, a copy of genomic information derived from tests done on their genomic material.

Thirdly, in at least some cases, additional genomic insights can be lost to the inaccessibility of the data processor. As one example, consider again a testing lab which tested a large portion of Bob's genomic sequence to screen for cardiac conditions. The test scope was for cardiac issues, that lab may be concerned only with the cardiac condition, and may not be interested in storing any other genomic data of Bob's—even though the test derived more genomic data than merely cardiac-related data. Although Bob performed this test, most of his data is lost, and if he later wishes to understand his genetic situation for other conditions, e.g. baldness, he has to perform additional testing to re-obtain this data. Alternatively, Bob can try to find the institution, if it still exists, and to request them to provide the genomic information, if they are still storing it, and if they are capable of providing this information externally for investigation in other contexts. The inefficiency of such a use of resources, inherent in such a situation, is evident. It is therefore advantageous, in some examples, to facilitate re-use of the genomic test results e.g. for other clinical needs.

Fourthly, in many cases metadata relating to the quality of reads of the genomic material are not saved, and are not available for the interpretation of Bob's genomic data.

There is thus a need for a solution to fully democratize genomic data, which allows patients and genetic test consumers to be in charge of their own genomic data, with the ability to carry their data with them from one institution to another, while making sure of data privacy, security and ownership.

As will be shown further herein with reference to FIGS. 1 to 5D, an alternative method and system for storage of genomic data can store data with increased security and improved capacity utilization.

A computerized data-storage system and is disclosed herein, with reference to FIGS. 3A-3B, which comprises a first processing circuitry. A computerized method is disclosed herein, with reference to FIGS. 1 to 2A and 5A to 5B, which comprises performing the following actions by the first processing circuitry:
a) receive a set of genomic data, comprising a plurality of items of loci-specific information. Each first item of loci-specific information comprises, at least, one or more encoded sequences, and/or one or more items of sequence metadata. Each first item of loci-specific information is associated with one or more items of identification information;
b) store, in one or more first storage locations, the set of genomic data;
c) store, in a mapping storage, a mapping between the item(s) of identification information and the corresponding first item of loci-specific information; and
d) store, in one or more second storage locations, item(s) of identification information, where the one or more second storage locations are associated with one or more individual instances of an organism.

The first storage location(s) and the storage location(s) are not identical.

Also, a computerized data-retrieval system and is disclosed herein, with reference to FIGS. 4A-4C, which comprises a second processing circuitry. A computerized method is disclosed herein, with reference to FIGS. 1 to 2A and 5C to 5D, which comprises performing the following actions by a second processing circuitry:
reconstructing at least a portion of an individual-specific instance of the set of genomic data,
where the individual-specific instance of the set is associated with one or more individual instances of the organism.

The individual-specific instance of the set comprises a sub-set of the plurality of first items of loci-specific information.

In some examples, these two systems and methods can facilitate an enhanced level of security of the individual-specific instance of the set of genomic data, as compared to a second level of security provided in a case of performing storage, in a single location, of the individual-specific instance of the set of genomic data and of the at least one item of identification information.

In some examples, this reconstruction comprises the following method:
e) receive the item(s) of identification information, from the at least one second storage location;
f) receive at least a portion of the set of genomic data, from the first storage location;
g) identify each first item of loci-specific information, based on the item(s) of identification information and on the mapping; and
h) output the portion(s) of the individual-specific instance of the set of genomic data.

In some non-limiting examples, the computerized data-storage and the computerized data-retrieval systems are the same.

In some non-limiting examples, the one or more encoded sequences comprise encoded sequences indicative of deviations from one or more genomic references, as will be disclosed forthwith.

Examples of the first storage location include a database stored at a genetic testing lab. Examples of the second storage location include a personal storage device such as a disk-on-key, which belongs to the client Bob. Additional disclosure concerning these locations is provided with reference to e.g. FIG. 3A, further herein.

Reverting again to FIG. 1, rather than, for example, storing the actual set of nucleotides in this portion of Bob's chromosome, a different structure is shown in the figure.

One or more genomic references 110, 115 are shown. These references can be either public references, and/or internal proprietary references belonging e.g. to the testing lab or other health institution. The use of transcripts is also possible.

Two shorter encodes sequences i101, i102 are shown, each comprising a portion of a genomic reference 110. Similarly, other encoded sequences are shown, which are indicative of deviations of variations from the one or more genomic references 110, 115. As one example, encoded sequence i103 contains the nucleotides AATTCCAGA. This represents a deviation from a portion of the sequence i101, which contains the nucleotides AATTCCACA. The deviation is that the second to last nucleotide in this sub-sequence is C in the reference, while it is G in the sequence i103. That is, in the two encoded sub-sequences there are different nucleotides in a particular position. The example shown is a single nucleotide polymorphism (SNP).

In a second example of deviation, sub-sequence i105 differs from its reference subsequence i102, in that GGCATTCAATAT_T is missing the second to last nucleotide, as compared to sub-sequence i102 which has T as the second to last nucleotide. In this sense, i103 is also referred to herein as a deviation sequence (or sub-sequence) of i101, and i105 is referred to herein as a deviation sequence of i102.

In some examples, these encoded sequences indicative of deviations from one or more genomic references or transcripts are referred to herein also as differences relative to the one or more genomic references/transcripts. Note also that the arrows connecting two encoded sequences and/or sub-sequences indicate that one is a deviation, or is otherwise derivative of, the other.

Variation/deviation data with respect to references can be, for example, representative of sequence variation or of structural variation.

For ease of exposition, encoded sub-sequences are referred to herein also as encoded sequences.

Note also that deviation sequences can themselves be sources of deviation sequences. For example, i111 is a deviation of i105, which is itself a deviation sequence of i102. An individual organism having deviation sequence i111 thus has an encoded sequence which includes the deviation indicated by i105, as well as the additional deviation indicated by i111. In this case, the individual has an encoded sequence i105, but with the additional deviation that the sub-sequence CAATAT of i105 is replaced by CATAAT. Note that deviation sequence i105 can be considered a reference with respect to deviation sequence i111.

Note also that i111 is a third non-limiting example of deviation, in which the order of nucleotides within a sequence/sub-sequence is different in a deviation sequence from the order of its reference.

A fourth example of deviation or variation is an insertion of one or more nucleotides. For example, i108 shows CATCT replacing CTCT in i106, where the A is inserted. Another example is translocation of an encoded sequence between chromosomes.

Thus, in one example, Bob's genomic sequence for this portion of his genome is indicated by the following set of pointers or identification codes: i109, i107, i111. This means that Bob's sequence can be reconstructed as follows: start with e.g. genomic reference 110. Bob's genomic sequence differs from that reference by all of the deviations indicated by pointers/codes i101 and i102. Bob's sequence further differs from i101 by the deviations indicated by i104. In at least this sense, i101 serves as a reference relative to i104. Bob's sequence further differs from i104 by the deviations i107 and i109. Bob's sequence further differs from i102 by the deviation i105. Bob's sequence further differs from i105 by the deviation i111. In some examples of the structure exemplified in the figure, this information can be derived by traversing the tree structure based on Bob's set of pointers or identification codes. In this sense, movement traversing the tree structure can conceptually be seen as “cascading” from level to level, comparing deviation sequence to its respective reference, and adding more and more differences (relative to the references) as each level is traversed.

More on pointers and ID codes is disclosed with reference to FIG. 2B further herein.

Note that since Bob's relevant genomic sequence is represented by the set of identification codes i109, i107, i111, this means that the deviations represented by the other codes associated with the structure, e.g. i103, i110, i314, i334, i106 and i108, are not relevant to Bob's genomics.

Of course, the example of the figure is simplified, showing only a small portion of genome, and only a small number of possible deviations from a reference, presented purely for exposition purposes.

Note also, that the example of the figure shows the deviations from references in a tree structure. This is non-limiting. Other structures or representations are possible.

Note also, that in the figure the deviation sequences are all sub-sequences of their respective references, that is they are shorter. In other examples, not shown in the figure, a deviation sequence is of the same length as its reference.

Bob is presented here as a non-limiting example of an individual instance of an organism. In this example, the organism is a human being. More generally, in some embodiments, the organism of the present disclosure may be at least one organism of the biological kingdom Animalia.

In more specific embodiments, such an organism may be any unicellular or multicellular invertebrate or vertebrate. More specifically, organisms from invertebrates may be an organism of the Phylum Porifera—Sponges, the Phylum Cnidaria—Jellyfish, hydras, sea anemones, corals, the Phylum Ctenophora—Comb jellies, the Phylum Platyhelminthes—Flatworms, the Phylum Mollusca—Molluscs, the Phylum Arthropoda—Arthropods, the Phylum Annelida—Segmented worms like earthworm and the Phylum Echinodermata—Echinoderms.

Still further, in some embodiments, the organism of the present disclosure may be any vertebrate organism, specifically, an organism derived from any of the vertebrates groups that include Fish, Amphibians, Reptiles, Birds and Mammals (e.g., Marsupials, Primates, Rodents and Cetaceans). In some particular embodiments, the methods of the present disclosure may be particularly applicable for any mammal (specifically, at least one of a human, Cattle, rodent, domestic pig (swine, hog), sheep, horse, goat, alpaca, lama and Camels), an avian, an insect, a fish, an amphibian, a reptile, a crustacean, a crab, a lobster, a snail, a clam, an octopus, a starfish, a sea-urchin, jellyfish, and worms.

In some other embodiments, the organism of the method of the present disclosure may be at least one organism of the biological kingdom Plantae. In some embodiments, any plants are applicable in the present disclosure.

In some examples the organism is a virus.

In some examples, the organism is a proband. The tree structure exemplified in the figure is a non-limiting example of a set of genomic data. The portion of Bob's genomic sequence exemplified in the figure is a non-limiting example of a portion of an individual-specific instance of the set of genomic data, where the individual is Bob.

Also, as indicated above, the figure exemplifies a set of genomic data which comprises only a portion of the genomic data of an organism (e.g. of humans). In other examples, the set of genomic data comprises genetic sequence data corresponding to an entire genome of the organism, e.g. the entire human genome.

Also, FIG. 1 exemplifies a case where one or more encoded sequences i105, i111, comprising encoded sequences, are indicative of deviations from one or more genomic references 110, 115. Recall that in some cases several references can be stored, since standard references have different revisions/updates, and different genomic tests are performed at different times along the timeline of a particular reference. Recall also that in some cases, the different versions of a reference influence the positions of particular segments.

Codes i101, i102 in the figure exemplify a possible implementation in which a genomic reference itself can be represented as a combination of several smaller/shorter sequences.

One segment can be represented in multiple unique ways in the system. This is exemplified in the figure by a single encoded sequence being represented by three different identification codes i101, i827, i881.

FIG. 2A-2B disclose a more general example and representation of a set of genomic data.

FIGS. 3A-4C disclose systems of storing such sets of genomic data, and of reconstructing at least a portion of an individual-specific instance of the set of genomic data (e.g. of Bob's set of genomic data).

FIGS. 5A-5D disclose methods of storing such sets of genomic data, and of reconstructing at least a portion of an individual-specific instance of the set of genomic data.

Attention is now drawn to FIG. 2A, schematically illustrating an example generalized view of a set of genomic data 210, in accordance with some embodiments of the presently disclosed subject matter. The figure illustrates a generalized architecture of the structure or format of a set of genomic data.

The non-limiting example set 210 of genomic data comprises n items 220, 223, 225 of loci-specific information. The term loci-specific information indicates that each item is associated with a particular locus, or with a plurality of particular loci, within an organism's genomic sequence. The items of loci-specific information are referred to herein also as first items, to distinguish them from other items disclosed herein.

Each item of loci-specific information comprises at least one of one or more encoded sequences and one or more items of sequence metadata. For example, Item 1 comprises one encoded sequence i, and the sequence meta-data items, a through m. For example, Item 2 comprises a plurality of encoded sequences, ii and iii. Note that Item 2 does not comprise items of sequence meta-data. By contrast, Item 3 comprises a meta-data item p, but does not comprise any encoded sequences.

The genomic sequences CATAAT and T_T, disclosed in FIG. 1 as being associated with codes i111 and i334, are non-limiting examples of encoded sequences. Note that least some of the encoded sequences can be of the same length, or of different lengths. For example, CATAAT comprises 6 nucleotides, while T_T comprises 3 nucleotide positions (where one position is empty). Examples of encoded sequences include DNA sequences and RNA sequences.

Sequence meta-data are items of data that relate to, describe, qualify or otherwise provide information on one or more encoded sequences. Non-limiting examples of sequence meta-data include the location of the sequence (e.g. location 70247901 on chromosome number 5, of interest in the Ashkenazi Jewish population), information related to the quality of a read of a particular segment or sequence by the testing equipment, the probe used in the genomic test, etc.

As shown in the figure, each item 220, 223, 225 of loci-specific information is associated with one or more items 228 of identification information. In the non-limiting example of the figure, Items 1 and 2 are each associated with an item 228 of identification information, specifically with identification code I and identification code II. An identification code is a non-limiting example of an item of identification information. Non-limiting examples of identification codes are disclosed in FIG. 2B.

In the figure, item n of loci-specific information is associated with a plurality of items of identification information, specifically with identification codes III and IV.

Although the items 228 of identification information I-IV are shown in the figure as being comprised in the set 210 of genomic data, in some examples they are stored separately. This is indicated by items of identification information being shown as dashed lines. In the case of, for example, FIG. 3 disclosed below, the set 210 of genomic data is stored in a first storage location, while the items 228 of identification information are stored in one or more storage locations, which are not identical to the storage locations.

Similarly, in some examples, the mapping between an item of loci-specific information and its associated/corresponding item(s) 228 of identification information, is stored together with the set of genomic data. In other examples, this mapping storage is in a location separate from the first storage location (in which the set 210 of genomic data is stored).

Note that a particular item(s) 228 of identification information can be associated with multiple individual instances 370 of an organism.

In some non-limiting examples, the set 210 of genomic data comprises one or more genomic references A and B, denoted by 230, 250. Such references are exemplified by genomic references 110, 115 of FIG. 1. In other examples, the genomic references are stored separately, not as part of the set of genomic data, either in the storage location, or in a different storage location.

Note that the storage of the set 210 as deviations from a reference, as disclosed for example with reference to FIG. 1, is a non-limiting example.

The set 210 of genomic data is referred to herein also as a first set 210 genomic data, to distinguish it from the second set 462 of genomic data, which is disclosed further herein e.g. with reference to FIG. 4.

Additional examples of items of identification information, and of the mapping between an item 220 of loci-specific information and its associated item(s) 228 of identification information, are disclosed with reference to FIG. 2B.

Attention is now drawn to FIG. 2B, schematically illustrating an example generalized view 200 of mapping, in accordance with some embodiments of the presently disclosed subject matter. The figure discloses non limiting examples of items 228 of identification information, of the mapping between an item 220 of loci-specific information and its associated item(s) of identification information, and of the mapping between items of identification information and clinical indications or other contexts of the request. As used in this disclosure, a context encompasses a particular clinical indication and the purpose of the particular test or report. A set 210A of genomic data is shown. It is exemplary of set 210 of genomic data, of FIG. 2A. For ease of exposition, arrows indicate the items 228 of identification information that are associated with each item of loci-specific information. These items of identification information are in some examples not stored in the set 210A, 210 of genomic data. In the example, pointer i334 is associated with the encoded sequence T_T, as indicated also in FIG. 1. Pointers i105, i107 and i109 are each associated with a corresponding particular encoded sequence. The details of those corresponding sequences are not shown in the figure. The code 120 is associated with an item of sequence metadata, in this case a Quality Score (QC) with a value of 0.9, which is associated with one or more encoded sequences (for example, those sequences were determined with a quality score of 0.9). Alternatively, “QC=0.9” may refer to more than one QC value. The code 123 is associated with another item of sequence metadata, in this case a probe identification “P7”, which is associated with one or more encoded sequences (for example, those sequences were obtained using Probe P7). Alternatively, “P7” may refer to more than one probe value.

Another non-limiting example of metadata is the test technology, test equipment vendor, and/or test methodology, used to obtain the genomic data. A further example is the time/date of the test. Note that each technology can have its technology-specific types of metadata.

Note that in some examples, a particular segment of Bob's genome is tested twice, at different times, using different technologies. In such a case, the system can store the relevant encoded sequence once, but store different metadata for each of the two tests.

In the above example, 120, 123, i334 etc. are pointers to data. For example, the pointer can indicate a particular location on a particular chromosome, i.e. within the genome.

Also shown are the metadata “QC=0.7”, which does not have a pointer, and the encoded genomic sequence CATCT, which also does not have a pointer.

Only a small portion of set 210A is shown, for ease of exposition only.

Also shown is a mapping storage 388. More on this storage is disclosed further herein with reference to FIG. 3A. This storage stores the mapping between items 228 of identification information and their associated items 220 of loci-specific information. The figure shows a number of non-limiting examples of how such mappings are stored, and what mapping data can look like. The person skilled in the art will readily see that other mapping possibilities exist. The non-limiting example of a table of mappings is shown.

Note that a particular item of loci-specific information 220, e.g. one particular genomic sequence, is in some examples associated with more than one item 228 of ID information. For example, the item of ID information, associated to a particular item 220, may be unique for each proband 370, and/or for each testing system/machine 373.

Pointer i334 is associated with, and directly mapped to, the encoded sequence T_T, as indicated also in FIG. 1. ID code 143 is mapped to pointer i109, which can be used to find the particular encoded sequence shown in set 210A. Similarly, ID code 145 is mapped to pointer 120.

ID code 150 is mapped to the pair of pointers 120 and 123, and thus is mapped to both the QC metadata and the probe metadata. By contrast, ID code 152 is mapped directly to the metadata value “QC=0.9”, without use of a pointer. Similarly, ID code 158 is directly mapped to the encoded sequence of nucleotides GTC, without use of a pointer.

ID code 160 maps to both an encoded sequence and to metadata. In the example of the figure, it maps to the encoded sequence using pointer i107, while it maps to metadata “QC=0.7” directly without use of a pointer. By contrast, ID Code 162 maps to encoded sequence and to multiple items of sequence metadata. The mapping to i107 and to “QC=0.7” is similar to that of code 160, while the mapping to the second item of metadata (“Probe=P7”) is done via a pointer 123.

ID code 163 maps to several sequence pointers. This is an example of associating one item of ID information with multiple items of loci-specific information, in this case with multiple encoded sequences. Similarly, ID 165 maps to several items of sequence metadata. Two such items are mapped via pointers 120 and 123, while the third, “QC=0.7”, is mapped directly. The mapping to multiple encoded sequences, or to multiple items of metadata, is indicated in the example of these records by dashes between the relevant items.

Example ID code 166 maps to other ID codes, 143 and 160, and via them to items of loci-specific information. Similarly, ID code 168 maps to multiple encoded sequences: to one via pointer i334, and to another via another ID code 143.

The last item 228 of identification is not an ID code. It is the non-limiting example of a hash, in this case with value 3FB45DA87. In the example of the figure, the item of identification information is a hash of various values, e.g. of various pointers, ID codes, encoded sequence values (such as GTT) and sequence metadata values (such as “Probe=P5”). In the specific example shown, it is a hash of pointer i334 and ID code 143.

In other examples, the item(s) of identification information comprises an encoded identification.

Note that pointers and identification codes are two non-limiting examples of items 228 of identification information.

Note that using mapping storage 388, if it is known that, for example, a particular item 143 of ID information is stored in a second storage location belonging to (or associated with) Bob, it can be determined that the pointer i109 is relevant to Bob, and thus that Bob's genome includes the encoded sequence CT__AT at the relevant locus.

The examples above exemplify cases where the stored mapping 388 maps the at least one item of identification information to at least one of the corresponding first item(s) of loci-specific information, the one or more encoded sequences, the one or more items of sequence metadata, at least one pointer to the corresponding first item of loci-specific information, and at least one other item of identification information.

Also shown is a second mapping storage 389. More on this storage is disclosed further herein with reference to FIG. 3A. The second mapping storage is used, in some examples, to associated items of identification information with clinical indications, particular applications, or other context information. The non-limiting example of a table of mappings is shown.

Non-limiting examples of clinical indications include a particular disease, e.g. cystic fibrosis, and/or a particular gene. Other examples include a predisposition to certain drugs, lifestyle risk factors, and determining a possible cause of a disease which a patient had or has. A non-limiting example of a context is a pre-conception screening, based on the data of the father and mother, where there is a need to determine the residual risk of a particular illness, given the genetics of the two parents. Note, in this regard, that all disclosure herein of sending a request regarding one individual, and storing retrieving and reconstructing data for one individual, applies as well to a situation of storing and reconstructing genetic data for a plurality of individuals, e.g. the father and mother in the above example.

Another non-limiting example of a context is receiving two segments of DNA, and determining the likelihood of their belonging to two relatives.

In the example of the figure, the ID code is mapped to the clinical indication CFTR (cystic fibrosis transmembrane conductance regulator), a gene coding a protein which is associated with the disease cystic fibrosis. Also in the example, two different ID codes 158 and 166 are mapped to SMN1, a gene associated with production of the survival motor neuron (SMN) protein. As will be shown further herein, this mapping can facilitate reconstruction, of portion(s) of an individual-specific instance of the set of genomic data which are associated with the specific clinical indications.

Note that the data structure 389 is presented as a non-limiting simplified example, for purposes of ease of exposition. Table 389 shows that codes 158 and 166 are both “related” to SMN1, with no more detail. In some other examples, the data structure, or perhaps a genetic professional, can indicate that both codes are “relevant” to SMN1, but that 158 is more relevant than 166.

In some examples, a particular item 220 of loci-specific information is associated with a single item i314 of identification information. In some other examples, particular item 228 of loci-specific information is associated with a plurality of items i101, i827, i881 of identification information. For example, the implementation may be such that Bob has pointer i103 associated with the encoded sequence AATTCCAGA, while Dave has a different pointer i882 associated with the same encoded sequence within the set 210 of genomic data.

FIGS. 3A-3B and 5A-5B disclose example systems and methods of storing sets of genomic data and items of identification, e.g. based on an individual-specific instance of the set of genomic data (e.g. of Bob's set of genomic data).

FIGS. 4A-4C and 5C-5D disclose example systems and methods of reconstructing at least a portion of an individual-specific instance of the set of genomic data (e.g. of Bob's set of genomic data), based on stored storing sets of genomic data and items of identification—as well as systems and methods of deriving item(s) of interpretive information associated with the individual instance of the organism (e.g. indicative of clinical indication(s)).

Attention is now drawn to FIG. 3A, schematically illustrating an example generalized schematic diagram 300 comprising a computerized genomic data storage system 305, in accordance with some embodiments of the presently disclosed subject matter. The diagram 300 illustrates, as well, example inputs and outputs of data storage system 305.

In some non-limiting examples, computerized genomic data storage system 305 includes a computer. It may, by way of non-limiting example, comprise a processing circuitry 310. This processing circuitry may comprise a processor 320 and a memory 330.

This processing circuitry 310 may be, in non-limiting examples, general-purpose computer(s) specially configured for the desired purpose by a computer program stored in a non-transitory computer-readable storage medium. They may be configured to execute several functional modules in accordance with computer-readable instructions. In other non-limiting examples, this processing circuitry 310 may be a computer(s) specially constructed for the desired purposes.

Example functional modules of processor 320 are disclosed further herein with reference to FIG. 3B.

In some examples, memory 330 of processing circuitry 310 is configured to store data associated with at least the analysis, extraction and encoding of features, and with storage of data, and various parameters and results disclosed with reference to the presently disclosed subject matter. For example, memory 330 can store the first and second mappings, before they are stored in 388 and 389. Similarly, memory 330 can store the collections of personal keys before they are stored in the individual's 370 storage device 395 etc.

In some examples, computerized genomic data storage system 305 comprises a first storage location 385. This location, in some examples, comprises a database or other data storage. This first storage location can be used to store the set 210, 210A of genomic data. If this set includes items of loci-specific information for a multiplicity of individual instances of an organism (e.g. multiple people, multiple dogs, or multiple tulips), e.g. storing genomic reference(s), transcripts, and a multiplicity of deviation sequences, as well as sequence metadata (in some examples), the set of genomic data can be referred to in some examples also as an aggregate database. This aggregated DB can store multiple features, each with its own logic and structure (e.g. not necessarily the structure exemplified by FIG. 1). In some examples, the data associated with hundreds, thousands or millions of individuals 370 are stored in this aggregated DB. The stored set 210 of data is such that genomic data of all of these individuals can be expressed in terms of at least a portion of the set of data.

In some examples, item(s) 220 of loci-specific information are records, e.g. of a database. Each item of information can be associated with one or more individuals. As one example of such, twins may share encoded sequences.

In some examples, if data of an entire genome is stored, a large portion of the data will be common to many or most of the individuals 370, with a somewhat smaller portion varying among the individuals.

In some examples, there is no need to store the references/transcripts, as they are not specific to an individual 370.

In some examples, computerized genomic data storage system 305 comprises a mapping storage 388. This location in some examples comprises a database or other data storage. This mapping storage 388 is referred to herein also as a first mapping storage 388, to distinguish it from second mapping storage 389. In some examples, this storage 388 stores mappings between item(s) 228 of identification information and corresponding first item(s) 220 of loci-specific information. e.g. as disclosed with reference to FIG. 2B.

In some examples, computerized genomic data storage system 305 comprises another mapping storage 389. This location in some examples comprises a database or other data storage. This mapping storage 389 is referred to herein also as a second mapping storage 389, to distinguish it from first mapping storage 388. In some examples, this storage 389 stores mappings between item(s) 228 of identification information and corresponding clinical indications or other context information, e.g. as disclosed with reference to FIG. 2B.

In some examples, computerized genomic data storage system 305 comprises a knowledge corpus 380. This location in some examples comprises a database or other data storage. This knowledge corpus 380 is referred to herein also as a first knowledge corpus 380, to distinguish it from second knowledge corpus 483 disclosed further herein with reference to FIG. 4A. In some examples, this first knowledge corpus 380 stores genomic knowledge, which can be used to facilitate extracting features from genomic data, creation of first item(s) 220 of loci-specific information, and creations of mappings between item(s) 228 of identification information and corresponding clinical indications or other context information.

In some examples, this knowledge corpus 380 holds e.g. quality data, which can in some cases be different per genetic testing technology utilized. For example, in one technology, there is “intensity data”, while another technology has ‘read depth”. In some examples, this knowledge corpus 380 holds metadata, used to determine confidence in the raw test data, and/or to analyze the relevant encoded sequence(s). Examples of this metadata include the location of the data, quality of the data, and frequency of that particular encoded sequence.

Examples of function of the first knowledge corpus are detailed further herein, with reference to FIGS. 3B and 5A.

Example schematic diagram 300 also depicts a genetic testing machine(s) 373. This machine performs genomic or genetic testing on genomic material samples obtained from an individual instance 370 of a biological organism, e.g. a proband, patient, or client 370, e.g. Bob. One or more such testing machines 373 can be operatively coupled to computerized genomic data storage system 305. In some examples, different testing machines 373 utilize different genetic testing technologies.

The genetic testing machine(s) 373 outputs 377 a genetic testing machine output 375, e.g. the results of the genomic test. This genetic testing machine output 375 is a non-limiting example of information indicative of a raw set of genomic data. This output 375 serves as an input 364 to the genomic data storage system 305, e.g. to the processor 320 of the processing circuitry 310. FIG. 3B provides more details on processor 320, and on how the input 364 is handled and processed.

This input 364, 375 to the processor can be of various formats. In some examples, the genetic testing machine output is 375 at least one of: a proprietary binary, a proprietary text, Comma delimited, tab delimited, a Variant Call Format (VCF) file, a genotype calling file, a FastQ® format file, a stream of data, or other formats.

In some examples, the genomic data storage system 305 outputs 366 one or more items of identification information 390 to one or more second storage locations 395. In the example of the figure, the second storage location 395 comprises at least one storage device associated with the organism. The specific example in the figure is a disk-on-key device 395 belonging to the proband Bob 370. In the example, the items of identification information 390 are stored in the format of a personal key data file 390, which is stored 393 on device 395. In some examples, the second storage location 395 is operatively coupled to the genomic data storage system 305.

The second storage location 395 is associated with at least one individual instance 370 of an organism. In the example, Bob is an individual instance, and the disk 395 belongs to him. In the example disclosed with reference to FIG. 1, the personal key data file 390 on Bob's personal disk 395 contains the set of pointers or identification codes i109, i107, i111. As disclosed with reference to FIG. 1, this information stored in second storage location 395 can be used, in some examples, to reconstruct at least a portion of an individual-specific (Bob's) instance of the set 210 of genomic data, e.g. a portion of Bob's genomic sequence.

In some examples, the at least one storage device is one of: local storage or on-line storage. Non-limiting examples of local storage 395 include a disk-on-key (as shown in the figure), a cellular phone, a computer hard-disk drive, and a tablet. Non-limiting examples of on-line storage 395 include the storage of an online provider and cloud storage.

In other examples, the second storage location 395 is associated with more than one individual instance of the organism, and items of identification information 390, for e.g. all of them, are stored at location 395. As one non-limiting example of this, disk-on-key 395 might store ID information of both Bob and his wife, and/or Bob and his children.

In some examples, each item 228 of identification information, stored in location 395, is associated with a corresponding individual instance 370. Also, each item of identification information is associated with an identification indication that is indicative of the corresponding individual instance 370 of the organism. A non-limiting example of such identification indication is an identification number.

For example, the ID information 228 of Bob may be associated with one identification number, e.g. his Social Security number, identifying him, while the ID information 228 of his wife may be associated with a different identification number, associated with her.

Such use of identification indications can, in some examples, facilitate the reconstruction of at least the portion of the individual-specific instance of the set of genomic data, which would correspond to the corresponding individual instance. Thus, assume for example in which a case Bob's Social Security number is ABC, and that number is associated with the set of pointers or identification codes i109, i107, i111. Bob's wife's Social Security number is XYZ, and that number is associated with a different set of pointers or identification codes i103, i111, 106. This information is all stored on the same shared disk or tablet 395. If the reconstruction process (disclosed further herein) accesses the disk 395 while requesting data for the identification indication XYZ, it will obtain Bob's wife's codes/pointers, and not those of Bob.

Note also that one individual instance 370 of an organism may be associated with more than one second storage location 395. For example, Bob may have his identification information 228 stored on both his cell phone 395 and on a disk-on-key 395.

An individual instance 370 of an organism is a specific example of an individual instance 370 of an entity. Thus, in some examples 395 is referred to herein as entity-specific storage location 395.

Note that in the figure, the first storage location 385 and the second storage location 395 are not identical.

In some examples, the set 210, 210A of genomic data, stored in first storage location 385, is stored in an encrypted format. In some examples, the items of identification information 228, 390, stored in second storage location 395, are stored in an encrypted format.

Attention is now drawn to FIG. 3B, schematically illustrating an example generalized schematic diagram of a processor 320, in accordance with some embodiments of the presently disclosed subject matter. The diagram 300 illustrates example functional modules of processor 320, which was disclosed with reference to FIG. 3A.

In some examples, processor 320 comprises input module 340. In some examples, this module is configured to receive information indicative of a raw set of genomic data, for example receiving genetic testing machine output 375 from e.g. Genetic Testing Machine 373. Note that the timing of the receipt of the data can vary. In one example, data indicative of an entire genome of a proband is received. In other examples, the data is received over time. Bob's data is received on Tuesday, and Carl's data is received a week later. Dan's data is received at two different points in time: the results of test A are received on one day, and the results of a different test B are received months, or even years later. Ed's data, related to certain chromosomes, is received at one point in time, while his data related to other chromosomes is received at another point in time.

In some examples, processor 320 comprises feature analyzer module 345. In some examples, this module is configured to analyze features of the information received by the input module 340. In some examples, the analyzing of the features is based on first knowledge corpus 380, which is associated with the set 210 of genomic data. Non-limiting examples of features that are analyzed include: encoding sequences, Quality Score (QC) data associated with a locus, epigenetic data, and vendor specific information. Non-limiting examples of Vendor specific information include R (intensity) & Theta (zygosity).

In some examples, processor 320 comprises one or more feature extractor modules 342, 344. In some examples, this module(s) is configured to extract one or more features from the received information, e.g. features analyzed by the feature analyzer module 345. In some examples there is a separate extractor module 342 per feature. The figure exemplifies this with n instances 342, 344 of the module, corresponding to n features. In some examples, for each of the n features there can exist zero or more instances of feature extractor module 342. In still other examples, one instance of feature extractor module 342 can extract multiple features. These features comprise encoded sequences and/or sequence metadata.

In some examples, processor 320 comprises one or more feature encoder modules 352, 354. In some examples, this module(s) is configured to encode the one or more features. The data is transformed into the relevant format(s), in which element of the data will be stored. This module can thereby generate each first item of loci-specific information and the at least one item 220, 223, 225 of identification information 228, i107. It thereby can generate the set 210, 210A of genomic data.

In some examples there is a separate encoder module 352 per feature. The figure exemplifies the case of n instances 352, 354 of the module, corresponding to n features.

In some examples, feature encoder module(s) 352, 354 generates the encoding sequences and the sequence metadata. It converts data into a different format, in which each element will be stored. In some examples, the module checks if a copy already exists. If it does not have an item of info of that value in the aggregated DB 385, it creates a new item. If, on the other hand, such an item already exists in the database, the module 352 could optionally create a new item/record with the same values, or could alternatively make use of the existing item.

In some examples, the new record in the first storage location 385 is sent via output module 359.

In some examples, feature encoder module(s) 352, 354 generates the mapping between the item(s) 228 of identification information and the corresponding item(s) 220 of loci-specific information. In some examples, the module(s) store this mapping in the first mapping storage 388. If the mapping storage is located external to the processor 320, in some examples the sending of the mapping to storage 388 is via output module 359.

In some examples, feature encoder module(s) 352, 354 are configured to generate the second mapping, between the item(s) 228 of identification information and corresponding clinical indication(s), for example. In some examples, the module(s) store this mapping in the second mapping storage 389. If the second mapping storage is located external to the processor 320, in some examples the sending of the mapping to storage 389 is via output module 359.

In some other examples, a separate module, other than feature encoder module 352, 354, performs the generation and storage of the second mapping.

In some examples, processor 320 comprises one or more personal keys encapsulator modules 357. In some examples, this module(s) is configured to encapsulate a collection of personal keys, comprising the at least one item of identification information. The storage of the item(s) 228 of identification information will in such a case comprise storing the collection of personal keys, e.g. in personal key data file 390 in second storage location 395. These encapsulated keys are in some examples based on items of identification information output by feature encoder module(s) 352, 354.

In some examples, collection of personal keys is sent to the second storage location 395 is carried out via output module 359.

In some examples, this encapsulator module 357 sets up all of the keys, for a particular individual 370, e.g. for Bob.

Note that an individual's collection of keys/items of ID information are unique to him or her, thus facilitating privacy.

In some examples, this module deletes the patient's unique collection of keys from memory 330, after they are output, e.g. for privacy/security reasons.

In some examples, processor 320 comprises one or more output modules 359. In some examples, this module(s) is configured to function as an interface between the processor and outside components, such as the first 385 and second 395 storage locations, and the first 388 and second 389 mapping storages.

More on the methods related to the system of FIGS. 3A-3B is disclosed further herein with reference to FIGS. 5A-5B.

Attention is now drawn to FIG. 4A, schematically illustrating an example generalized schematic diagram 400 of data retrieval and interpretation, in accordance with some embodiments of the presently disclosed subject matter. The diagram 400 illustrates a computerized data-retrieval system 410 and a computerized data-interpretation system 460. The diagram 400 illustrates, as well, example inputs and outputs of these systems 410, 460.

In some non-limiting examples, computerized genomic data retrieval system 410 includes a computer. It may, by way of non-limiting example, comprise a processing circuitry 420. This processing circuitry may comprise a processor 430 and a memory 425.

This processing circuitry 420 may be, in non-limiting examples, general-purpose computer(s) specially configured for the desired purpose by a computer program stored in a non-transitory computer-readable storage medium. They may be configured to execute several functional modules in accordance with computer-readable instructions. In other non-limiting examples, this processing circuitry 420 may be a computer(s) specially constructed for the desired purposes.

Example functional modules of processor 430 are disclosed further herein with reference to FIG. 4B.

In some examples, memory 425 of processing circuitry 420 is configured to store data associated with at least the receipt of requests 407, and the retrieval and matching of items 228 of identification information and items 220 of loci-specific information, as well as various parameters and results disclosed with reference to the presently disclosed subject matter. For example, memory 330 can store: lists of ID codes or pointers retrieved from user device 395, retrieved items 220 of loci-specific information, clinical indication information in stakeholder requests 407, items 228 of ID information which correspond to the clinical indications etc. Similarly, in some cases the memory 425 is configured to store the individual-specific 462 instance of the set of genomic data, before it is sent to interpretation system 460.

This processing circuitry, processor and memory are referred to herein also as second processing circuitry 420, second processor 430 and second memory 425, to distinguish them from first processing circuitry 310, first processor 320 and first memory 330 of genomic Data Storage System 305, disclosed with reference to FIG. 3A.

In some examples, computerized genomic data retrieval system 410 comprises a mapping storage 488. This location in some examples comprises a database or other data storage. This mapping storage 488 is referred to herein also as a first mapping storage 488, to distinguish it from second mapping storage 489. In some examples, this storage 488 stores mappings between item(s) 228 of identification information and corresponding first item(s) 220 of loci-specific information. e.g. as disclosed with reference to FIG. 2B. In some examples, this storage 488 is identical to the first mapping storage 388, disclosed with reference to FIG. 3A. For example, system 410 can, in some implementations, instead access the storage 388 on system 305. This possibility is illustrated by the dashed or broken lines.

In some examples, computerized genomic data retrieval system 410 comprises second mapping storage 489. This location, in some examples, comprises a database or other data storage. In some examples, this storage 489 stores mappings between item(s) 228 of identification information and corresponding clinical indications or other context information, e.g. as disclosed with reference to FIG. 2B. In some examples, this storage 489 is identical to the second mapping storage 389, disclosed with reference to FIG. 3A. For example, system 410 can in some implementations instead access the storage 389 on system 305. This possibility is illustrated by the dashed or broken lines.

The depiction of genomic data storage system 305 in diagram 400, as operatively coupled with system 410, is to indicate that in some cases the mapping storages 488, 489 reside on system 305, e.g. as storages 388, 389. In such a case, retrieval system 410 communicates with storage system 305, to access the mapping storages 388, 389.

In some non-limiting examples, computerized genomic data interpretation system 460 includes a computer. It may, by way of non-limiting example, comprise a processing circuitry 470. This processing circuitry may comprise a processor 480 and a memory 475.

This processing circuitry 470 may be, in non-limiting examples, general-purpose computer(s) specially configured for the desired purpose by a computer program stored in a non-transitory computer-readable storage medium. They may be configured to execute several functional modules in accordance with computer-readable instructions. In other non-limiting examples, this processing circuitry 470 may be a computer(s) specially constructed for the desired purposes.

Example functional modules of processor 480 are disclosed further herein with reference to FIG. 4B.

In some examples, memory 475 of processing circuitry 470 is configured to store data associated with at least the receipt of requests 407, and the derivation and output of items 409 of interpretation information, as well as various parameters and results disclosed with reference to the presently disclosed subject matter. For example, memory 330 can store all or some the following: the individual-specific 462 instance of the set of genomic data, received from retrieval system 410, items 409 of interpretation information derived based on checking with second knowledge corpus 483 (before they are output 409 to the external device(s) 405).

This processing circuitry, processor and memory are referred to herein also as third processing circuitry 470, third processor 480 and third memory 475, to distinguish them from first processing circuitry 310, first processor 320 and first memory 330 of genomic data storage system 305, disclosed with reference to FIG. 3A, and from second processing circuitry 420, second processor 430 and second memory 425 of genomic data storage system 410.

In some examples, computerized genomic data interpretation system 460 comprises a knowledge corpus 483. This corpus in some examples comprises a database or other data storage. This knowledge corpus 483 is referred to herein also as a second knowledge corpus 483, to distinguish it from first knowledge corpus 380. In some examples, this second knowledge corpus 483 stores information that can be utilized to derive interpretations of the reconstructed portion(s) of an individual-specific instance of the set of genomic data 210, 210A. In one example, the secondary corpus stores the clinical significances and impacts of variations in the genomic sequence. Examples of function of the second knowledge corpus are detailed further herein, with reference to FIGS. 4C and 5D.

In some examples, computerized genomic data interpretation system 460 comprises access permissions datastore 490. This location in some examples comprises a database. In some examples, this access permissions datastore stores permissions per user/proband/patient, for accessing their genomic data. Further disclosure of this datastore appears further herein.

In some examples, the genomic data retrieval system 410 and the genomic data interpretation system 460 are located on the same system, e.g. sharing a single processing circuitry. This possibility is indicated by the dashed lines around processing circuitry 470.

Example schematic diagram 400 also depicts an external stakeholder system(s) or device(s) 405. Non-limiting illustrative examples of such systems include computer systems associated with stakeholders of genomic data, such as e.g. a physician, a genetic counselor at a genetic counseling clinic or other facility, a hospital, a health care system, a genetic test laboratory, another health facility, an employer, an insurer, or some other institution. Such parties often have a need to obtain genomics-related information of a particular proband or other individual, for example to obtain or determine their risk of certain diseases with a genetic component. In some examples, external stakeholder system 405 is operatively coupled with system 410 and/or with system 460.

In the example of the figure, the external system 405 sends a request 407 for an interpretive report, or for other interpretive information, to data retrieval system 410, and receives the interpretive report or other information from data interpretation system 460. In other non-limiting examples, the service architecture is different: the system 405 sends request 407 to interpretation system 460, as well as receiving the report from system 460. In such an example, data retrieval system 410 functions as a back-end for data interpretation system 460.

Example schematic diagram 400 also depicts the proband, patient or other individual 370, e.g. Bob. In some examples, individual 370 interacts with data interpretation system 460 to set access permissions for his or her data. In some cases, the access permissions are specific to the stakeholder system, and are specific to certain clinical indications. For example, Bob may allow a heart clinic to access his genomic data that is related to heart disease, but not to baldness. As another example, Bob may allow a physician's office X to access all or some of his genomic data, while not permitting another physician's office Y to access any of the data.

Example schematic diagram 400 also depicts first storage location 385 and second storage location 395, disclosed with reference to FIG. 3A. As part of the reconstruction of the portion(s) of an individual-specific instance of the set 210 of genomic data, in some examples retrieval system 410 is operatively coupled to, and accesses, these two storage locations 385, 395, to obtain items 220 of loci-specific information and items 228 of identification information. Note that, although both first storage location 385 and genomic data storage system 305 are depicted in the figure, in some examples first storage location 385 is in fact part of genomic data storage system 305, e.g. as depicted in FIG. 3A.

An example scenario of retrieving and interpreting portion(s) of an individual-specific instance of the set of genomic data 210, 210A, utilizing the systems disclosed with reference to this FIG. 4A, are disclosed with reference to FIGS. 4B and 4C.

Example schematic diagram 400 also depicts the genomic data storage system 305, e.g. disclosed with reference to FIG. 3A.

Attention is now drawn to FIG. 4B, schematically illustrating an example generalized schematic diagram of a processor 430, in accordance with some embodiments of the presently disclosed subject matter. The diagram illustrates example functional modules of processor 430, which was disclosed with reference to FIG. 4A.

In some examples, processor 430 comprises clinical indications matching module 437. In some examples, this module is configured to receive clinical indication information, indicative of one or more clinical indications associated with the individual instance 370 of the organism, or to receive other information indicative of the context of the retrieval of the genomic information. In one example, this is the request 407 for an interpretive report, or for other interpretive information, sent e.g. from the external system 405. In another example, the clinical indication information is received from the request input module 481 of genomic data interpretation system 460, which in turn received the request 407 from the external system 405.

In some examples, clinical indications matching module 437 is configured, instead of or in addition to the above, to identify, based at least on the received clinical indication information and on the second mapping 389, 489, one or more corresponding items 228 of identification information. This derived corresponding item(s) 228 of identification information is referred to herein also as a mapped item 228 of identification information, or as a mapped identification code 228.

As a non-limiting example, per that disclosed in FIG. 2B, the matching module 437 receives clinical indication information, indicative of clinical indication CFTR, and, using mapping storage 389, derives the identification code 145. In another example the clinical indication information is indicative of CFTR and baldness.

In some examples, the identifying or deriving of corresponding item(s) 145 of identification information, based at least on the received clinical indication information and on the second mapping 389, 489, comprises performing a lookup of the at least one item 145 of identification information, e.g. in the mapping table 389.

In some examples, processor 430 comprises identification items input module 432. In some examples, this module is configured to obtain or receive one or more items of identification information 228, 145, i334, pointers, or ID codes. In some examples this information is obtained from second storage location 395 associated with the individual 370.

In some examples the receiving of items of ID information comprises receiving all items of identification information associated with the individual instance(s) 370 of the organism. In the example disclosed above with reference to Bob, the module retrieves or otherwise receives all of the three pointers i109, i107, i111 associated with Bob, as they are all of the items of identification information associated with Bob.

In some other examples, not all items of identification information associated with the instance(s) 370 are received. Rather, only a portion or strict sub-set of all of the individual's items of identification information are received. For example, the clinical indication received by clinical indications matching module 437 may be for SMN1, which maps (in FIG. 2B) to identification codes 158, 166. The individual's 370 storage device 395 contains code 158, but not 166. The code 166 is not associated with the individual's genomics, and thus is not obtained. Similarly, in this example, the individual 370 is also associated with identification items i105, 165, 168, etc., but these are not obtained, since they are not associated with the requested clinical indication SMN1.

In another example, the input module 432 is requested to retrieve Bob's ID information items, as they relate only to chromosome number 13, and thus any ID codes associated with others of Bob's chromosomes are not retrieved.

In another example, module 432 retrieves all of the items of ID information on second storage location 395, but then filters out those that are not relevant for the currently requested interpretation. In this sense, the module 432 can be said to obtain relevant item(s) 166 of identification information, from the received item(s) 228, 166 of identification information. This obtaining of relevant item(s) 166 of identification information is based on the corresponding item(s) 158, 166 of identification information derived by clinical indications matching module 437 based on the second mapping. The relevant item(s) 166 of identification information thus constitutes the item(s) 166 of identification information, for purposes of further processing of these items 166 of identification information.

In some examples, processor 430 comprises data matching module 435. In some examples, this module is configured to match one or more items of identification information 228, 145, i334 with one or more items 220, 225 of loci-specific information. In some examples this is performed by the module accessing first mapping storage 388, 488. For example, in the example of FIG. 2B, assuming that the identification information item obtained by identification items input module 432 is code 158, the first mapping in mapping storage 388 indicates that the item 220 of loci-specific information to retrieve from the set 210 of genomic data (stored in first storage location 385) is the encoded genomic sequence GTC. In another example, the ID information obtained is code 166. The mapping storage 388 indicates that 166 maps to codes 143 and 160. The mapping storage in turn indicates that these two codes map to pointer i109, which point to an encoded sub-sequence, and to pointer i107 and the sequence metadata value QC=0.7.

In some examples, the identifying of the first item(s) 220, 225 of loci-specific information, based on items(s) 228 of identification information and on the mapping, which is performed by data matching module 435, comprises performing a lookup of item(s) 228 of identification information, e.g. in a table in first mapping storage 388, 488.

In some examples, processor 430 comprises loci-specific information input module 434. In some examples, this module is configured to receive at least a portion of the set 210, 210A of genomic data, e.g. by accessing first storage location 385.

In some non-limiting examples, the module receives the entire set 210 of genomic data, and not just a portion of the set, from the aggregate database or other first storage location 385.

In some other non-limiting examples, the receiving of the at least a portion of the set 210 of genomic data utilizes the received at least one item 228 of identification information. For example, as indicated in the earlier example, the encoded sequences pointed to by pointers i109 and i107 are received.

Note also that in some examples, the portions of the set of genomic data to receive are determined by data matching module 435, based on the mapping storage 388, 488. In turn, in some implementations this first mapping is based on those items 228 of identification information that correspond to the received clinical indication information, which in turn were determined (in some implementations) based on the clinical indications matching module 437, which consults the second mapping storage 389, 489.

That is, in such a case the systems 410 and 460 will retrieve and reconstruct only those portions of the individual's genome which are relevant to the context of the stakeholder 405 request 407.

Other non-limiting example implementations are possible. In one such example, all of Bob's 370 items 228 of identification information are read by module 432 from second storage location 395, and all items 220 of loci-specific information are read by module 434 from the set 210 of genomic data in first storage location 385. Data matching module 435 is then used to match up items of ID information and items of loci-specific information, based on the first mapping 388, and the sub-set of items 220 within the genomic data set 210 are obtained, based on this matching.

In still another example, all of all of Bob's 370 items 228 of identification information are read by module 432 from second storage location 395. Data matching module 435 is then used to match up items of ID information and items of loci-specific information, based on the first mapping 388. Then module 434 retrieves only the relevant sub-set of items 220 within the genomic data set 210, from first storage location 385, based on this matching.

Note that in some of the above examples only items 220 of loci-specific information which correspond to Bob's items 228 of ID information are obtained. However, in some implementations, loci-specific information input module 434 must still retrieve additional 220 of loci-specific information. For example, in the implementation of FIG. 1, and as disclosed above with reference to Bob, the module 434 retrieves or otherwise receives the encoded sequences which correspond to the three pointers i109, i107, i111 associated with Bob. However, these encoded sequences are not sufficient to reconstruct Bob's sequence. Thus, module 434 will retrieve, as well, the sequences pointed to by i104, i101, i105, i102, since these sequences are “parent” or “reference” sequences relative to the “child” sequences which appear relatively lower in the figure. That is, the module will traverse up the tree structure, starting from the sequences associated with the ID information stored in storage device 395, to obtain all encoded sequence information required for the reconstruction.

Note, in this regard, that in some examples the module 434 also retrieves the relevant reference sequences or transcripts 110, 115, so as to facilitate the reconstruction.

In some examples, processor 430 comprises data-set reconstruction module 439. In some examples, this module is configured to reconstruct at least a portion of an instance 462 of the set 210 of genomic data that is specific to an individual 370. In the non-limiting example of FIG. 1, the tree structure is traversed, and the individual's 370 deviation sequences (exemplifying items of loci-specific information) are applied to their reference sequences, as well as associating sequences their sequence metadata. In one non-limiting example, the reconstruction yields the encoded sequence of all, or part, of Bob's 370 chromosome 19, along with metadata associated with the sequence.

In some examples, the reconstruction portions 462 of an individual-specific instance of the set 210 of genomic data, is referred to herein also as “second items” 462 of information, to distinguish them from the first items 220 of loci-specific information which compose the set 210 of genomic data. The set of second items 462, relevant to individual 370, is also referred to herein as a second set of genomic data.

In some examples, module 439 is also configured to output the reconstructed portion(s) 462, of the individual-specific instance 462 of the set 210 of genomic data, e.g. to computerized genomic data interpretation system 460. In other examples, the reconstructed instance 462 is output to another system, e.g. to stakeholder system 405.

Note that if set 210 includes genomic data that is associated with a plurality of probands or other individuals, the individual-specific instance 462 of the set 210 is in many cases smaller than the entire set.

Attention is now drawn to FIG. 4C, schematically illustrating an example generalized schematic diagram of a processor 480, in accordance with some embodiments of the presently disclosed subject matter. The diagram illustrates example functional modules of processor 480, which was disclosed with reference to FIG. 4A.

In some examples, processor 480 comprises request input module 481. In some examples, this module is configured to receive clinical indication information, indicative of one or more clinical indications associated with the individual instance 370 of the organism, or to receive other information indicative of the context of the retrieval of the genomic information. In one example, this is the request 407 for an interpretive report, or for other interpretive information, sent e.g. from the external system 405. In some examples, this clinical indication information is then forwarded to clinical indications matching module 437 of genomic data retrieval system 410.

In some examples, processor 480 comprises access control module 482. In some examples, this module is configured to determine whether requests 407 will be processed, based on access permissions. In some examples, this module is configured to determine whether outputs 409 of items of interpretive information will be provided to external systems 405, based on access permissions. For example, the output is performed in response to receipt of an authorization indication, which indicates that the particular external system 405 is authorized to receive 409 item(s) of interpretive information. In some examples, the authorization indication is associated with the individual instance(s) 370 of the organism. In some examples, the authorization indication is indicative of consent of the individual instance 370 of the organism.

In some examples the authorization indication is a record, located in a list or other datastore of access permissions, not shown in FIG. 4. In some examples, the authorization indication is a configurable parameter.

In one non-limiting example of the above, the list indicates that systems of Hospital A are not allowed at all to access the systems 410 and/or 460. The list indicates that Cancer Hospital B is permitted to access the systems, only for a certain set of clinical indications associated with cancer. Hospital B is not authorized, however, to access the systems regarding other contexts, e.g. proband height or eye color, not related to cancer. That is, access per stakeholder and/or per individual 370 can be context-specific, in some cases.

As another example, for the individual instance Bob 370, the configuration in the list is that Hospital B cannot access his data, but Hospital C can access his data. For a different individual instance 370 Carl, the configuration data for access authorization is such that Hospital C can access only his genomic data related to cardiac clinical indications, while Counseling Clinical can access all of his genomic data.

In some examples, processor 480 comprises interpretations module 484. In some examples, this module 484 is configured to determine whether requests 407 will be processed, based on access permissions. In some examples, this module is configured to receive the portion(s) 462 of the individual-specific instance of the set of genomic data, which were generated by the computerized data-retrieval system 410, and which were output by it.

In some examples, this module 484 is configured to derive one or more items 409 of interpretive information associated with the individual instance 370 of the organism. In some examples this derivation is based at least on the reconstructed portion(s) 462 of the individual-specific instance of the set 210, 210A of genomic data. In this way, after the individual's genomic information has been reconstructed, the system 460 can derive meaning from it. In some examples, the item(s) of interpretive information is indicative of one or more clinical indications associated with the individual instance 370 of the organism.

Examples of clinical indications include the individual 370 being at risk for certain medical conditions (e.g. a disease), the individual's existing or potential children having a certain level genetic risk for a medical condition (based on the genetic data of the parent 370), and ethnicity/ancestry information associated with the tested individual 370.

For example, the system determines that Bob's genomic data indicates that he is at an increased risk of developing a particular type of cancer, or of having children with a certain genetic condition.

A clinical indication is one type of “context” of the interpretation. Thus, more generally, system 460 can derive, and output items of interpretive information that are indicative of one or more contexts.

In some examples, the deriving of the interpretive information is based on a second knowledge corpus 483, shown in FIG. 4A. This knowledge corpus stores information relating genomic data to various contexts. In some cases, a genomic variation has a clinical significance, e.g., benign, pathogenic etc. The information from the second knowledge corpus can be used to generate genetic test reports, e.g. related to the clinical significance.

For example, the corpus 483 can indicate that the encoded sequence T_T, pointed to by i334, is indicative of a particular medical condition, or that the combination of the two sequences ATA (pointed to by i314) and CATCT (pointed to by i108) is indicative of a 10% increase in the probability of developing another medical condition. That is, the second corpus 483 can be utilized to determine clinical significance of certain genomic data of the individual 370.

In some examples, the first knowledge corpus 380 and the second knowledge corpus 483 share at least certain items of information. In some other non-limiting examples, one knowledge corpus 380, 483 is stored, containing information that is configured for use by both feature analyzer module 345 and interpretations module 484.

In some examples, processor 480 comprises output module 486. In some examples, this module 484 is configured to output 409 item(s) of interpretive information to one or more external system(s) 405, e.g. belonging to stakeholders of the set 210 of genomic data, e.g. genetic counseling clinics and hospitals. In some examples, the output of the item(s) of interpretive information comprises a report. In some examples, this module is also referred to herein as interpretations output module 486, to distinguish it from other output modules disclosed herein.

In some, examples, responsive to the outputting 409 of item(s) of interpretive information, the output module 484, or another module, deletes the reconstructed individual-specific instance 462 of the set of genomic data from the computerized data-interpretation system 460. Similarly, in some, examples, responsive to the outputting of the portions of the individual-specific instance 462 of the set 210 of genomic data, and/or in response to the outputting 409 of item(s) of interpretive information, the module 439, or another module, deletes the reconstructed individual-specific instance 462 of the set of genomic data from the computerized data-retrieval system 410. This can be done, for example, to facilitate increased security and privacy of the user's 370 personal genomic data.

Note that, for ease of exposition only, the storage system 305, the retrieval system 405 and the interpretation system 460 are shown in FIGS. 3A-4C as three separate systems, one function per system. Different distribution of functions across computers system are possible. In one such example, data storage system 305 and data retrieval system 405 are combined. In another such example, data retrieval system 405 and data interpretation system 460 are combined. In still another example, data storage system 305 and data interpretation system 460 are combined, serving as a “front end” to testing machines 373 and external stakeholder systems 405, while data retrieval system 405 functions as a “back end” system. In still another example, the functions of all three systems 305, 410, 460 are combined into one system.

Similarly, other physical and logical arrangements of storage and databases are possible. As one example of this, in some cases the first mapping storage 388 and the first storage location 385 are located at the same physical location. Thus, for example, any combination of the functionalities of the first storage location 385, first mapping storage 388, 488, first mapping storage 389, 489, first knowledge corpus 380, and second knowledge corpus 483 are possible.

Note, however, that second storage location 395 should be separate from first storage location 385, to meet security concerns.

In some examples, the storages 385, 388, 488, 389, 489, 380, 483 stores data that is relatively more persistent than the data stored in memories 330, 425, 475. The examples of FIGS. 3A-4C are non-limiting. In other examples, other divisions of data storage between the various storages and memories 330, 425, 475 may exist.

FIGS. 3A-4C illustrate only a general schematic of the system architecture, describing, by way of non-limiting example, certain aspects of the presently disclosed subject matter in an informative manner, merely for clarity of explanation. It will be understood that the teachings of the presently disclosed subject matter are not bound by what is described with reference to FIGS. 3A-4C.

Only certain components are shown, as needed, to exemplify the presently disclosed subject matter. Other components and sub-components, not shown, may exist. Systems such as those described with respect to the non-limiting examples of FIGS. 3A-4C may be capable of performing all, some, or part of the methods disclosed herein.

Each system component and module in FIGS. 3A-4C can be made up of any combination of software, hardware and/or firmware, as relevant, executed on a suitable device or devices, which perform the functions as defined and explained herein. The hardware can be digital and/or analog. Equivalent and/or modified functionality, as described with respect to each system component and module, can be consolidated or divided in another manner. Thus, in some embodiments of the presently disclosed subject matter, the system may include fewer, more, modified and/or different components, modules and functions than those shown in FIGS. 3A-4C. To provide one non-limiting example of this, in some examples results interpretations module 484 and output module 486 are combined. Similarly, in some examples feature analyzer module 345 and feature extractor module 342, 244 are combined. Similarly, in some examples, there may be separate output modules 359 for each destination storage location 385, 395.

One or more of these components and modules can be centralized in one location, or dispersed and distributed over more than one location, as is relevant. In some examples, the computerized genomic data storage system 305, the computerized data-retrieval system 410, and/or computerized data-interpretation system 460, utilize a cloud implementation, e.g. implemented in a private or public cloud.

Each component in FIGS. 3A-4C may represent a plurality of the particular component, possibly in a distributed architecture, which are adapted to independently and/or cooperatively operate to process various data and electrical inputs, and for enabling operations related to a computerized hearing test. In some cases, multiple instances of a component may be utilized for reasons of performance, redundancy and/or availability. Similarly, in some cases, multiple instances of a component may be utilized for reasons of functionality or application. For example, different portions of the particular functionality may be placed in different instances of the component.

Communication between the various components of the systems of FIGS. 3A-4C, in cases where they are not located entirely in one location or in one physical component, can be realized by any signaling system or communication components, modules, protocols, software languages and drive signals, and can be wired and/or wireless, as appropriate. The same applies to interfaces such as output modules 359, 486.

Before disclosing example process flows, with reference to FIGS. 5A-5D, some example technical advantages are presented.

In some examples, the security of the genomic data, and the privacy of that data as it related to the individual 370, are increased. Firstly, if a hacker or thief, or other malicious party, steals/obtains Bob's disk on key or other storage device 395, all they know is “i109, i107, i111”. This set of codes or other values has no meaning per se. The malicious party has no knowledge of any of Bob's genomic data, as they have no way to cross-reference the ID information 228 with items 220 of loci-specific information. By contrast, in a case where actual encoded sequences were stored on the storage device, the thief has direct access.

Secondly, consider a hacker or other party who breaks into or otherwise accesses first storage location 385. All they have is a large list of sequences and metadata, with no connection to individuals. The same applies to a lab or other institution which accesses first storage location 385. The set 210 of genomic data, that is the actual “content” of the genomic information of individuals, is in effect anonymized. Only aggregate data, e.g. “encoded sequence TCAA at locus XYZ”, is stored, and that piece of data can be associated with any number of individuals-one, dozens, thousands or millions.

In the example architecture and method disclosed herein, in order to understand what Bob's genomic data is, there is a need to have access to all three of first storage location 385, second storage location 395, and first mapping storage 388, 488. Unlike current methods and systems, there is no one “single point of failure”, one location where sufficient data is stored that permits knowledge of Bob's genomic information.

The proposed architecture and method thus can facilitate an enhanced level of security of the individual-specific instance of the set of genomic data, as compared to a second level of security provided in a different case—in a case of a reconstruction based on performing storage, in a single location, of the individual-specific instance of the set of genomic data.

In some examples, the first mapping storage 388, 488 is stored in a location separate from first storage location 385. Similarly, in some examples, the second mapping storage 389, 489 is stored in a location separate from first storage location 385. Either or both of these options can further increase the security of the solution.

In addition, in some examples, the first storage location 385, first mapping storage 388, 488 and/or second mapping storage 389, 489 are controlled (e.g. are owned) by an institution, company or other body which is distinct from stakeholders. For example, these storages, and data retrieval system 410, reside at Company M, which is separate from the hospitals/labs/physicians' offices/genetic counselors.

Not only do the stakeholder systems 405 not have access to Bob's genomic data at a single location, there is also an additional layer of security. The stakeholder systems 405 are not capable of accessing any portion of the genomic data 210 itself, nor the mapping storages 388, 389, 488, 489. They can only send requests 407 indicative of e.g. clinical indications or other contexts. They receive the report or other form of items of interpretation information 409, that is the meaning or interpretation of genomic information, but not the genomic information itself. That is, the external systems 405 lack direct access of to the individual-specific instance 462 of the set of genomic data.

The report is given in the context of the particular query. For example, these systems are told that Bob has a 10% increased chance of baldness, as compared to the general population, but they are not told that Bob has encoded sequence GTT at a particular locus and sequence CATGA at another specific locus.

In addition, in some examples, also data interpretation system 460 resides at Company M (or at another Company N), which is separate from the hospitals/labs/physicians' offices/genetic counselors. These stakeholders in some examples have no control over the access permissions datastore 490.

An additional layer of security exemplified in FIG. 4B is the use of access permissions, in some examples, specific to combinations of individual 370, stakeholder 405 and clinical indications/contexts. For example, a cancer clinic is not permitted to receive baldness-related interpretation information regarding Bob, but only cancer-related interpretation information 409, since such consent was not configured in the access permissions data store 490.

Note that in the example of FIGS. 4A-4B, a plurality of systems and locations, each with its own security measures, are required to function together to provide the interpretive information: the data-interpretation system 460, data retrieval system 410 including the first and/or second mappings, the first storage location 385 and the second storage location 395.

In some cases, any or all of the above advantages provide improved protection of Personal Health Information (PHI).

In addition, in some examples, the architecture and methods disclosed herein can facilitate access to the portion(s) 462 of the individual-specific instance of the set of genomic data, while utilizing a reduced amount storage, as compared to a second storage amount required in a different case—in a case of performing storage, in a single location, of individual-specific instances of the set of genomic data for each individual organism of a plurality of organisms.

In one example, the storage of a full genome for one human requires approximately ⅓ terabyte (TB) of storage space, not including metadata. When adding metadata, the storage requirement can in some cases increase to about 1-2 TB per person.

In many cases, this is too much information to store in personal storage devices 395 of each individual 370. On the other hand, storage of this data per individual, for hundreds or thousands of individuals, at an institution such as a hospital or a laboratory, is in some cases inefficient, since, in many cases, there is similarity of portions of genomic material across many individuals. The method disclosed herein can facilitate storage of one copy of a particular item 220 of loci-specific information, associate it with one or more items 228 of identification information, and store these items 228 of ID information in the storage devices 395 associated each individual.

This can in some cases provide storage efficiency, e.g. where the data is stored on large scales (a large number of individuals and/or large portions of their genomes). In at least some cases, the larger the number of individual organisms for which data is stored, the greater are the storage efficiencies.

In effect, the method herein can provide a form of compression, and of encryption, for genomic data. This compression is lossless, since the reconstruction method enables reconstruction of all of the relevant data, without loss.

Note that in some implementations, it may be decided to store copies of items 220 of loci-specific information for each individual. Even in such a case, the system and method disclosed herein can provide the security and privacy advantages disclosed above.

In some examples there is a third type of technical advantage. Testing data acquired by a particular genomic test is not lost. After the test is performed, e.g. to identify a particular clinical indication(s) or other context, the acquired data is stored in first storage location 385, and is available for use in the future when receiving interpretation requests 407 related to the same or other context. In some cases, it is possible to derive interpretations related to different contexts, without requiring performance of an additional test.

In addition, as more tests are performed on Bob, each capturing data related to somewhat different portions of his genome (in some cases with some overlap of encoded sequences), comprehensive information on Bob's genome is accumulated.

FIGS. 5A-5D provide detailed flows of the computerized method or process 500 for storage, retrieval and interpretation of genomic data.

Attention is now drawn to FIGS. 5A to 5B, illustrating one example generalized flow chart diagram, of a flow of a process or method, for storage of genomic data, in accordance with certain embodiments of the presently disclosed subject matter. This process is, in some examples, carried out by systems such as those disclosed with reference to FIG. 3.

The flow starts at 505. According to some examples, information 375, indicative of a raw set of genomic data, is received (block 505). This is done, in some examples, by input module 340 of processor 320, of processing circuitry 310 of computerized genomic data storage system 305.

According to some examples, features of the received information 375 are analyzed (block 510). This is done, in some examples, by feature analyzer module 340 of processor 320.

According to some examples, one or more features from the received information 375 are extracted (block 515). This is done, in some examples, by feature extractor module(s) 342, 344 of processor 320.

According to some examples, one or more features from the received information 375 are encoded (block 517). This is carried out, in some examples, by feature encoder module(s) 352, 354 of processor 320. In some cases, this encoding thereby generates first item(s) 220 of loci-specific information and item(s) 228 of identification information.

According to some examples, a collection of personal keys are encoded (block 519). This is carried out, in some examples, by personal keys encapsulator module 357 of processor 320. In some cases, this collection comprises the one or more items 228 of identification information. In some examples, this collection is in the form of a personal key data file 390.

Note that blocks 505-519 are enclosed in a dashed line box 508. Box 508 is one example of a process for generating items 220 of loci-specific information and generating and encapsulating items 228 of identification information.

The flow continues A to FIG. 5B.

According to some examples, the set 210 of genomic data is stored in at least one first storage location 385 (block 520). This is done, in some examples, by feature encoder module(s) 352, 354 sending the information via output module 359. In some examples, the first storage location 385 is aggregated DB 385. In some examples, the stored set 210 of genomic data comprises the items 220 of loci-specific information.

According to some examples, a mapping, between the item(s) 228 of identification information and the corresponding first item(s) 220 of loci-specific information, is stored (block 524). This is carried out, in some examples, by feature encoder module(s) 352, 354, sending the information via output module 359. In some examples, the storage is in mapping storage 388, 488.

According to some examples, a second mapping, between the item(s) 228 of identification information and one or more clinical indications or other contexts, is stored (block 526). This is carried out, in some examples, by feature encoder module(s) 352, 354, sending the information via output module 359. In some examples, the storage is in second mapping storage 389, 489.

According to some examples, the item(s) 220 of loci-specific information is stored in at least one second storage location 395 (block 527). This is carried out, in some examples, by feature encoder module(s) 352, 354, or by personal keys encapsulator module 357, sending the information via output module 359. In some examples, the second storage location(s) 395 is associated with one or more individual instances 370 of an organism, e.g. the human proband Bob 370. In some examples, the stored information is in the form of personal key data file 390, comprising the collection of personal keys.

Note that blocks 520-527 are enclosed in a dashed line box 528. Box 528 is one example of storing items 220 of loci-specific information and generating and encapsulating items 228 of identification information, and related mappings.

Note that in the non-limiting example of FIGS. 3 and 4, the steps within boxes 508 and 528 are performed utilizing genomics data storage system 305.

The flow continues from FIG. 5B to FIG. 5C.

Attention is now drawn to FIGS. 5C to 5D, illustrating one example generalized flow chart diagram, of a flow of a process or method, for retrieval and interpretation of genomic data, in accordance with certain embodiments of the presently disclosed subject matter. This process is, in some examples, carried out by systems such as those disclosed with reference to FIG. 4.

According to some examples, the item(s) 228 of identification information are received (block 530). This is carried out, in some examples, by identification items input module 432, of processor 430, of processing circuitry 420 of computerized genomic data storage system 410. In some examples, the items 228 are received from the storage device(s) 395 or other second storage location(s) 395.

According to some examples, clinical indication information, or other context information, is received (block 531). This is performed, in some examples, by clinical indications matching module 437, of processor 430. In another example, this step is performed by request input module 481 of processor 480, of processing circuitry 470 of genomics data interpretation system 460, which, for example, forwards the information to module 437.

In some examples, this clinical indication information is indicative of one or more clinical indications associated with the individual instance 370 of the organism. In one example, the clinical indication information is contained in stakeholder request(s) 407, received from a stakeholder system 405. One non-limiting example of a clinical indication is SMN1. Another example is a genetic counseling clinic sending a request to determine residual risk for one or more illnesses, when performing pre-conception screening for parents. Another example is a police query to determine if Bob committed a certain crime, e.g. whether the DNA on a piece of evidence is his. Another example is an ethnicity analysis of Bob. Still another example is a lifestyle analysis: e.g. whether Bob is more likely to do well with a high-endurance physical training program or a high-intensity physical training program.

In other examples, block 531 comprises receiving other information indicative of the context of retrieval of the genomic information.

According to some examples, corresponding item(s) 228 of identification information are identified (block 532). This is performed, in some examples, by clinical indications matching module 437, of processor 430. In some examples, this identifying is based on the received clinical indication information and on the second mapping. This second mapping is stored, for example, in second mapping storage 489, 389, located on genomic data retrieval system 410 and/or on genomic data storage system 305.

For example, all items of identification information that are mapped to the clinical indication are identified. In the examples disclosed with reference to FIG. 4B, identification codes 158, 166 correspond to the clinical indication SMN1.

Note also that different health institutions, and different national systems, may investigate different loci on the genome when trying to determine the presence of a clinical indication, such as cystic fibrosis or eye color. Therefore, in some implementations of the example of FIG. 2B, block 532 will identify code 158 for the US health system, while identifying code 166 for the French health system, since each health system considers encoded sequences of different loci when investigating, for example, SMN1.

According to some examples, a relevant item(s) of identification information is obtained (block 533). This is performed, in some examples, by clinical indications matching module 437. This is done, for example, by matching the identified corresponding item(s) of identification information, derived by the second mapping, with the item(s) 228 of identification information obtained from the proband's 370 associated second storage location 395.

As exemplified with reference to FIG. 4B, in one case the proband's 370 storage device 395 contains identification items 158, i105, 165, 168, but only code 158 is a corresponding item of identification information, since the second mapping shows that 158 is associated with the requested clinical indication SMN1. In this example, code 158 is the obtained relevant item 228 of identification information.

According to some examples, at least a portion of the set 210, 210A of the genomic data is received (block 535). This is performed, in some examples, by clinical indications matching module 437. In some cases, this data is received from the first storage location 385. In some cases, this portion of the set of genomic data comprises a plurality of first items 220 of loci-specific information.

According to some examples, relevant first item(s) of loci-specific information are identified (block 537). This is performed, in some examples, by data matching module 435. In some examples, the identification is performed, at least based on the item(s) of identification information (identified e.g. at block 533), and on the first mapping. This first mapping is stored, for example, in first mapping storage 488, 388. In some examples, the first storage is located on genomic data retrieval system 410 and/or on genomic data storage system 305.

In examples where, in blocks 531, 532, 533, the clinical indication(s) was used to identify relevant item(s) 220 of identification information, those relevant items will constitute the item(s) of identification information used in block 537 for the matching using the first mapping, and thus the relevant first item(s) of loci-specific information.

In some examples, this block results in, or facilitates, a reconstruction of at least a portion of an individual-specific instance of the set 210 of genomic data, e.g. a portion of Bob's 370 genomic data (encoded sequences and/or sequence metadata).

According to some examples, at least a portion 462 of the individual-specific instance of the set of genomic data is output 462 (block 540). This is performed, in some examples, by data matching module 435. In other examples, it is output by a separate module, not shown in FIG. 4B. In the non-limiting example of FIG. 4A, the instance 462 is output to genomic data interpretation system 460.

According to some examples, the reconstructed portion 462, of the individual-specific instance of the set of genomic data, is deleted (block 545). This is performed, in some examples, by data matching module 435, deleting it from system 410 after it is output in step 540. In other examples, it is output by a separate module, not shown in FIG. 4B. In other examples, the reconstructed portion 462 is deleted from data retrieval system 410, only at step 570 below (or in parallel with that step), after the output of the item(s) of interpretation information.

Note that blocks 530-545 are enclosed in a dashed line box 528. Box 538 is one example of a process of retrieving items 220 reconstructing and outputting a least a portion of an instance 261 of the set of genomic data, associated with one or more individuals 370.

The flow continues C to FIG. 5D.

According to some examples, an authorization indication is received (block 550). This is performed, in some examples, by access control module 481, of processor 480 of processing circuitry 470 of computerized genomic data interpretation system 460. In some examples, the authorization indication is associated with individual instance(s) 370 of the organism. In some examples, the authorization indication indicates that a requesting external system(s) 405 is authorized to receive 409 item(s) of interpretive information. In some examples, the authorization indication is indicative of consent of the individual instance 370 of the organism.

According to some examples, at least a portion 460 of the individual-specific instance of the set of genomic data is received (block 550). This is performed, in some examples, by interpretations module 482, of processor 480. In some other examples, processor 480 has a separate input module (not shown) to handle this input.

In some examples, this portion 460 of the individual-specific instance of the set of genomic data was output 460 by the computerized data-retrieval system 410 at block 540, which reconstructed it at block 537.

In some examples, receipt of this portion 460 of the individual-specific instance of the set of genomic data, is performed only in response to receipt of an authorization indication in block 550.

According to some examples, item(s) of interpretive information, associated with the individual instance 370 of the organism, are derived (block 560). This is performed, in some examples, by interpretations module 482. In some examples, these item(s) of interpretive information are indicative of one or more clinical indications. In some examples, the derivation is based at least on the reconstructed portion(s) 462 of the individual-specific instance of the set 210 of genomic data. In some examples, the derivation is performed only in response to receipt of an authorization indication in block 550.

According to some examples, item(s) of interpretive information are output 409 (block 565). This is performed, in some examples, by interpretations output module 486. In some examples, these item(s) of interpretive information are output to one or more external stakeholder systems/devices 405. In some examples, the outputting to an external system is performed only in response to receipt of an authorization indication in block 550.

According to some examples, the reconstructed individual-specific instance of the set of genomic data is deleted (block 570). This is performed, in some examples, by output module 486, or by interpretations module 482. In some examples, the deletion is from the computerized data-retrieval system 410 and/or from the computerized data-interpretation system 460. In some examples, this deletion is performed responsive to deriving of the interpretive information. In some examples, this deletion is performed responsive to the outputting of the item(s) of interpretive information.

Note that blocks 550-570 are enclosed in a dashed line box 558. Box 558 is one example of a process deriving and outputting items of context-specific interpretative information, based on a reconstructed individual-specific instance of the set of genomic data, or on a portion of that instance.

Note that in the non-limiting example of FIGS. 3 and 4, the steps within boxes 538 and 558 are performed utilizing genomics data retrieval system 410 and genomics data interpretation system 460.

Note that the above description of processes 500, 508, 528, 538, 558 is a non-limiting example only.

In some embodiments, one or more steps of the flowchart exemplified herein may be performed automatically. The flow and functions illustrated in the flowchart figures may for example be implemented in systems 305, 410, 460 and in processing circuitries 310, 420, 470, and may make use of components described with regard to FIGS. 3 and 4. It is also noted that whilst the flowchart is described with reference to system elements that realize steps, such as for example systems 305, 410, 460, and processing circuitries 310, 420, 470, this is by no means binding, and the operations can be carried out by elements other than those described herein.

It is noted that the teachings of the presently disclosed subject matter are not bound by the flowcharts illustrated in the various figures. The operations can occur out of the illustrated order. One or more stages illustrated in the figures can be executed in a different order and/or one or more groups of stages may be executed simultaneously. For example, steps 565 and 570, shown in succession, can be executed substantially concurrently, or in a different order.

Similarly, some of the operations or steps can be integrated into a consolidated operation, or can be broken down into several operations, and/or other operations may be added. As a non-limiting example, in some cases blocks 531, 532 and/or 533 can be combined.

In embodiments of the presently disclosed subject matter, fewer, more and/or different stages than those shown in the figures can be executed. As one non-limiting example, certain implementations may not include blocks 531, 532, 526, or 545.

In the claims that follow, alphanumeric characters and Roman numerals, used to designate claim elements such as components and steps, are provided for convenience only, and do not imply any particular order of performing the steps.

It should be noted that the word “comprising” as used throughout the appended claims, is to be interpreted to mean “including but not limited to”.

While there has been shown and disclosed examples in accordance with the presently disclosed subject matter, it will be appreciated that many changes may be made therein without departing from the spirit of the presently disclosed subject matter.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter may be, at least partly, a suitably programmed computer. Likewise, the presently disclosed subject matter contemplates a computer program product being readable by a machine or computer, for executing the method of the presently disclosed subject matter, or any part thereof. The presently disclosed subject matter further contemplates a non-transitory machine-readable or computer-readable memory tangibly embodying a program of instructions executable by the machine or computer for executing the method of the presently disclosed subject matter or any part thereof. The presently disclosed subject matter further contemplates a non-transitory computer readable storage medium having a computer readable program code embodied therein, configured to be executed so as to perform the method of the presently disclosed subject matter.

Those skilled in the art will readily appreciate that various modifications and changes can be applied to the embodiments of the invention as hereinbefore described without departing from its scope, defined in and by the appended claims.

1. A computerized data-storage system, comprising a processing circuitry, configured to perform the following method:
a) receive a set of genomic data, comprising a plurality of first items of loci-specific information,
wherein each first item of loci-specific information of the plurality of items of loci-specific information comprises at least one of one or more encoded sequences and one or more items of sequence metadata,
wherein each first item of loci-specific information is associated with at least one item of identification information;
b) store, in at least one first storage location, the set of genomic data;
c) store, in a mapping storage, a mapping between the at least one item of identification information and the corresponding first item of loci-specific information; and
d) store, in at least one second storage location, the at least one item of identification information, where the at least one second storage location is associated with at least one individual instance of an organism,
wherein the at least one first storage location and the at least one second storage location are not identical;
the method thereby facilitating a reconstruction, of at least a portion of an individual-specific instance of the set of genomic data,
the reconstruction being performed by a computerized data-retrieval system,
the individual-specific instance of the set being associated with at least one individual instance of the organism, the individual-specific instance of the set comprising a sub-set of the plurality of first items of loci-specific information,
the method thereby facilitating an enhanced level of security of the individual-specific instance of the set of genomic data, as compared to a second level of security provided in a case of performing storage, in a single location, of the individual-specific instance of the set of genomic data and of the at least one item of identification information.

2. The computerized data-storage system of claim 1, wherein the reconstruction comprises performing the following method:
d) receive the at least one item of identification information, from the at least one second storage location;
e) receive at least a portion of the set of genomic data, from the first storage location;
f) identify the each first item of loci-specific information, based on the at least one item of identification information and on the mapping; and
g) output the at least the portion of the individual-specific instance of the set of genomic data.

3. The computerized data-storage system of any one of claims 1 and 2, wherein the one or more encoded sequences comprising encoded sequences indicative of deviations from one or more genomic references.

4. The computerized data-storage system of claim 3, wherein the set of genomic data further comprising the one or more genomic references.

5. The computerized data-storage system of any one of claims 1 to 4, wherein the receiving of the at least a portion of the set of genomic data utilizes the received at least one item of identification information.

6. The computerized data-storage system of claim 5, wherein said step (c) further comprising storing, in a second mapping storage, a second mapping between the at least one item of identification information and at least one clinical indication.

7. The computerized data-storage system of claim 6, wherein in said step (d) the receiving of the at least one item of identification information further comprises performing the following steps:
(1) receiving clinical indication information, indicative of at least one clinical indication associated with the individual instance of the organism;
(2) identifying, based on the received clinical indication information and on the second mapping, a corresponding at least one item of identification information;
(3) obtaining a relevant at least one item of identification information, from the received of the at least one item of identification information, based on the corresponding at least one item of identification information, the relevant at least one item of identification information constituting the at least one item of identification information.

8. The computerized data-storage system of any one of claims 1 to 7, wherein the stored mapping maps the at least one item of identification information to at least one of: the corresponding first item of loci-specific information; the one or more encoded sequences; the one or more items of sequence metadata; a pointer to the corresponding first item of loci-specific information; at least one other item of identification information.

9. The computerized data-storage system of any one of claims 2 to 8, wherein the receiving the at least a portion of the set of genomic data, from the first storage location, comprises receiving the set of genomic data.

10. The computerized data-storage system of any one of claims 2 to 9, wherein the receiving the at least one item of identification information associated with the at least one individual instance of the organism, from the at least one second storage location, comprises receiving all items of identification information associated with the at least one individual instance of an organism.

11. The computerized data-storage system of any one of claims 1 to 10, wherein the at least one item of identification information comprises at least one identification code.

12. The computerized data-storage system of any one of claims 1 to 11, wherein the at least one item of identification information comprises at least one of a hash and an encoded id.

13. The computerized data-storage system of any one of claims 1 to 12, wherein the mapping storage and the first storage location are located at the same location.

14. The computerized data-storage system of any one of claims 1 to 13, wherein at least some encoded sequences are of lengths different from each other.

15. The computerized data-storage system of any one of claims 1 to 14, wherein the organism is one of a unicellular organism, a multicellular organism and a virus.

16. The computerized data-storage system of claim 15, wherein the organism is a human.

17. The computerized data-storage system of claim 16, wherein the organism is a proband.

18. The computerized data-storage system of any one of claims 1 to 17, wherein the set of genomic data comprises genetic sequence data corresponding to an entire genome of the organism.

19. The computerized data-storage system of any one of claims 1 to 18, wherein the method further comprises performing, prior to said step (a), the following steps to generate the set of data:
h) receiving information indicative of a raw set of genomic data
i) analyzing features of the received information;
j) extracting one or more features from the received information; and
k) encoding the one or more features, thereby generating the each first item of loci-specific information and the at least one item of identification information.

20. The computerized data-storage system of any one of claims 1 to 19, wherein the information indicative of a raw set of genomic data is a genetic testing machine output associated with the individual instance of the organism,
the method therefore facilitating re-use of the results for other clinical needs.

21. The computerized data-storage system of claim 20, wherein the genetic testing machine output is at least one of: a proprietary binary, a proprietary text, Comma delimited, tab delimited, a Variant Call Format (VCF) file, a genotype calling file, a FastQ® format file, a stream of data, another format.

22. The computerized data-storage system of any one of claims 19 to 21, wherein in said step (i) the analyzing of the features is based on a first knowledge corpus associated with the set of genomic data.

23. The computerized data-storage system of any one of claims 19 to 22, wherein the features comprise at least one of: the encoding sequence; Quality Score (QC) data associated with a locus; epigenetic data; vendor specific information.

24. The computerized data-storage system of any one of claims 12 to 23, the method further comprising performing, prior to said step (c):
l) encapsulating a collection of personal keys, comprising the at least one item of identification information,
wherein the storing of the at least one item of identification information comprising storing the collection of personal keys.

25. The computerized data-storage system of any one of claims 1 to 24, wherein the enhanced level of security comprises a lack of direct access of external systems to the individual-specific instance of the set of genomic data.

26. The computerized data-storage system of any one of claims 1 to 25, the method thereby facilitating access to the at least the portion of the individual-specific instance of the set of genomic data, while utilizing a reduced storage amount, as compared to a second storage amount required in a case of performing storage, in a single location, of individual-specific instances of the set of genomic data for each individual organism of a plurality of organisms.

27. The computerized data-storage system of any one of claims 1 to 26, wherein a particular item of loci-specific information is associated with a single item of identification information.

28. The computerized data-storage system of claims 1 to 27, wherein a particular item of loci-specific information is associated with a plurality of items of identification information.

29. The computerized data-storage system of any one of claims 1 to 28, wherein the at least one second storage location is associated with more than one individual instance of an organism,
wherein each item of identification information is associated with a corresponding individual instance of the more than one individual instance of the organism,
wherein the each item of identification information is associated with an identification indication that is indicative of the corresponding individual instance of the organism,
thereby facilitating the reconstruction of the at least the portion of the individual-specific instance of the set of genomic data in correspondence to the corresponding individual instance.

30. The computerized data-storage system of claim 29, wherein the identification indication is an identification number.

31. The computerized data-storage system of any one of claims 1 to 30, wherein the at least one second storage location comprises at least one storage device associated with the organism.

32. The computerized data-storage system of claim 31, wherein the at least one storage device is one of: local storage or on-line storage.

33. The computerized data-storage system of any one of claims 1 to 32, wherein the at least one item of identification information is stored in one or more personal data key files at the at least one second storage location.

34. A computerized data-retrieval system, comprising a processing circuitry, configured to perform the following:
a) provide a set of genomic data, comprising a plurality of first items of loci-specific information,
wherein each first item of loci-specific information of the plurality of items of loci-specific information comprises at least one of an encoded sequence and sequence metadata,
wherein each first item of loci-specific information is associated with at least one item of identification information;
wherein the set of genomic data was generated by performance of the following method by a computerized data-storage system:
(i) store, in at least one first storage location, the set of genomic data;
(ii) store, in a mapping storage, a mapping between the at least one item of identification information and the each first item of loci-specific information; and
(iii) store, in at least one second storage location, the at least one item of identification information, where each second storage location is associated with at least one individual instance of an organism,
wherein the at least one first storage location and the at least one second storage location are not identical;
b) reconstruct at least a portion of an individual-specific instance of the set of genomic data,
the individual-specific instance being associated with the at least one individual instance of an organism, the individual-specific instance comprising a sub-set of a plurality of first items of loci-specific information,
wherein the reconstruction comprises the following method:
(i) receive at least a portion of the set of genomic data, from the first storage location;
(ii) receive the at least one item of identification information, from the at least one second storage location;
(iii) identify the each first item of loci-specific information, based on the at least one item of identification information and on the mapping; and
(iv) output the at least the portion of the individual-specific instance of the set of genomic data,
the method thereby facilitating an enhanced level of security of the individual-specific instance of the set of genomic data, as compared to a second level of security provided in a case of a reconstruction based on performing storage, in a single location, of the individual-specific instance of the set of genomic data and of the at least one item of identification information.

35. The computerized data-storage system of claim 34, wherein the one or more encoded sequences comprising encoded sequences indicative of deviations from one or more genomic references.

36. The computerized data-retrieval system of any one of claims 34 to 35, wherein the identifying the at least one first item of loci-specific information, based on the at least one item of identification information and on the mapping, comprises performing a lookup of the at least one item of identification information.

37. The computerized data-retrieval system of any one of claims 34 to 36, further configured to perform the following:
(v) responsive to the outputting of the at least the portion of the individual-specific instance of the set of genomic data, delete the reconstructed individual-specific instance of the set of genomic data from the computerized data-interpretation system.

38. A computerized data-interpretation system, comprising a processing circuitry, configured to perform the following:
(A) receive the output of the at least the portion of the individual-specific instance of the set of genomic data, generated by the computerized data-retrieval system of any one of claims 34 to 36; and
(B) derive at least one item of interpretive information associated with the individual instance of the organism, based at least on the reconstructed at least a portion of the individual-specific instance of the set of genomic data.

39. The computerized data-interpretation system of claim 38, wherein the at least one item of interpretive information is indicative of at least one clinical indication associated with the individual instance of the organism.

40. The computerized data-interpretation system of any one of claims 38 to 39, wherein the deriving of the interpretive information is based on a second knowledge corpus.

41. The computerized data-interpretation system of any one of claims 38 to 40, the system further configured to perform the following:
(C) output the at least one item of interpretive information to at least one external system.

42. The computerized data-interpretation system of claim 41, wherein the output of the at least one item of interpretive information comprises a report.

43. The computerized data-interpretation system of any one of claims 41 to 42, wherein the at least one of external system is associated with at least one of a physician, a genetic counselor, a health care system, a genetic test laboratory, an employer, and an insurer.

44. The computerized data-interpretation system of any one of claims 38 to 43, further configured to perform the following:
(D) responsive to one of the deriving of the interpretive information and the outputting of the at least one item of interpretive information, delete the reconstructed individual-specific instance of the set of genomic data from at least one of the computerized data-retrieval system and the computerized data-interpretation system.

45. The computerized data-interpretation system of any one of claims 38 to 44, wherein the outputting of the at least one item of interpretive information to the external system in said step (c) is performed in response to receipt of an authorization indication which indicates that the at least one external system is authorized to receive the at least one item of interpretive information.

46. The computerized data-interpretation system of claim 45, wherein the authorization indication is associated with the at least one individual instance of the organism.

47. The computerized data-interpretation system of any one of claims 45 to 46, wherein the authorization indication is indicative of consent of the individual instance of the organism.

48. A computerized method, capable of being performed by a computerized data-storage system comprising a processing circuitry, the method comprising performing the following actions:
a) receive a set of genomic data, comprising a plurality of first items of loci-specific information,
wherein each first item of loci-specific information of the plurality of items of loci-specific information comprises at least one of an encoded sequence and sequence metadata,
wherein each first item of loci-specific information is associated with at least one item of identification information;
b) store, in at least one first storage location, the set of genomic data;
c) store, in a mapping storage, a mapping between the at least one item of identification information and the corresponding first item of loci-specific information; and
d) store, in at least one second storage location, the at least one item of identification information, where the at least one second storage location is associated with at least one individual instance of an organism,
wherein the at least one first storage location and the at least one second storage location are not identical;
the method thereby facilitating a reconstruction, of at least a portion of an individual-specific instance of the set of genomic data,
the reconstruction being performed by a computerized data-retrieval system,
the individual-specific instance of the set being associated with at least one individual instance of the organism, the individual-specific instance of the set comprising a sub-set of the plurality of first items of loci-specific information,
the method thereby facilitating an enhanced level of security of the individual-specific instance of the set of genomic data, as compared to a second level of security provided in a case of performing storage, in a single location, of the individual-specific instance of the set of genomic data and of the at least one item of identification information.

49. A computerized method, capable of being performed by a computerized data-retrieval system comprising a processing circuitry, the method comprising performing the following actions:
a) provide a set of genomic data, comprising a plurality of first items of loci-specific information,
wherein each first item of loci-specific information of the plurality of items of loci-specific information comprises at least one of an encoded sequence and sequence metadata,
wherein each first item of loci-specific information is associated with at least one item of identification information;
wherein the set of genomic data was generated by performance of the following method by a computerized data-storage system:
(i) store, in at least one first storage location, the set of genomic data;
(ii) store, in a mapping storage, a mapping between the at least one item of identification information and the each first item of loci-specific information; and
(iii) store, in at least one second storage location, the at least one item of identification information, where each second storage location is associated with at least one individual instance of an organism,
wherein the at least one first storage location and the at least one second storage location are not identical;
b) reconstruct at least a portion of an individual-specific instance of the set of genomic data,
the individual-specific instance being associated with the at least one individual instance of an organism, the individual-specific instance comprising a sub-set of a plurality of first items of loci-specific information,
wherein the reconstruction comprises the following method:
(i) receive the at least one item of identification information, from the at least one second storage location;
(ii) receive at least a portion of the set of genomic data, from the first storage location;
(iii) identify the each first item of loci-specific information, based on the at least one item of identification information and on the mapping; and
(iv) outputting the at least the portion of the individual-specific instance of the set of genomic data,
the method thereby facilitating an enhanced level of security of the individual-specific instance of the set of genomic data, as compared to a second level of security provided in a case of a reconstruction based on performing storage, in a single location, of the individual-specific instance of the set of genomic data.

50. A non-transitory computer readable storage medium tangibly embodying a program of instructions that, when executed by a computerized data-storage system, cause the computer to perform a computerized method, the method being performed by a processing circuitry of the computerized data-storage system and comprising performing the following actions:
a) receive a set of genomic data, comprising a plurality of first items of loci-specific information,
wherein each first item of loci-specific information of the plurality of items of loci-specific information comprises at least one of an encoded sequence and sequence metadata,
wherein each first item of loci-specific information is associated with at least one item of identification information;
b) store, in at least one first storage location, the set of genomic data;
c) store, in a mapping storage, a mapping between the at least one item of identification information and the corresponding first item of loci-specific information; and
d) store, in at least one second storage location, the at least one item of identification information, where the at least one second storage location is associated with at least one individual instance of an organism,
wherein the at least one first storage location and the at least one second storage location are not identical;
the method thereby facilitating a reconstruction, of at least a portion of an individual-specific instance of the set of genomic data,
the reconstruction being performed by a computerized data-retrieval system,
the individual-specific instance of the set being associated with at least one individual instance of the organism, the individual-specific instance of the set comprising a sub-set of the plurality of first items of loci-specific information,
the method thereby facilitating an enhanced level of security of the individual-specific instance of the set of genomic data, as compared to a second level of security provided in a case of performing storage, in a single location, of the individual-specific instance of the set of genomic data and of the at least one item of identification information.

51. A non-transitory computer readable storage medium tangibly embodying a program of instructions that, when executed by a computerized data-retrieval system, cause the computer to perform a computerized method, the method being performed by a processing circuitry of the computerized data-retrieval system and comprising performing the following actions:
a) provide a set of genomic data, comprising a plurality of first items of loci-specific information,
wherein each first item of loci-specific information of the plurality of items of loci-specific information comprises at least one of an encoded sequence and sequence metadata,
wherein each first item of loci-specific information is associated with at least one item of identification information;
wherein the set of genomic data was generated by performance of the following method by a computerized data-storage system:
(i) store, in at least one first storage location, the set of genomic data;
(ii) store, in a mapping storage, a mapping between the at least one item of identification information and the each first item of loci-specific information; and
(iii) store, in at least one second storage location, the at least one item of identification information, where each second storage location is associated with at least one individual instance of an organism,
wherein the at least one first storage location and the at least one second storage location are not identical;
b) reconstruct at least a portion of an individual-specific instance of the set of genomic data,
the individual-specific instance being associated with the at least one individual instance of an organism, the individual-specific instance comprising a sub-set of a plurality of first items of loci-specific information,
wherein the reconstruction comprises the following method:
(i) receive the at least one item of identification information, from the at least one second storage location;
(ii) receive at least a portion of the set of genomic data, from the first storage location;
(iii) identify the each first item of loci-specific information, based on the at least one item of identification information and on the mapping; and
(iv) outputting the at least the portion of the individual-specific instance of the set of genomic data,
the method thereby facilitating an enhanced level of security of the individual-specific instance of the set of genomic data, as compared to a second level of security provided in a case of a reconstruction based on performing storage, in a single location, of the individual-specific instance of the set of genomic data.
